(19) United States
(12) Patent Application Publication
(10) Pub. No.: US 2009/0112100 A1
(43) Pub. Date: Apr. 30, 2009

(54) BIOLOGICAL INFORMATION COLLECTING DEVICE AND METHOD FOR CONTROLLING THE SAME

(75) Inventor: Masahiko SHIOI, Osaka (JP)

Correspondence Address: MCDERMOTT WILL & EMERY LLP, 600 13TH STREET, NW, WASHINGTON, DC, 20005-3096, US

(73) Assignee: PANASONIC CORPORATION, Osaka (JP)

(21) Appl. No.: 12/347,489

(22) Filed: Dec. 31, 2008

Related U.S. Application Data
(63) Continuation of application No. PCT/JP2008/050861, filed on Jan. 23, 2008.

(30) Foreign Application Priority Data
Jan. 24, 2007 (JP) 2007-013313

(51) Int. Cl. A61B 1/227; A61B 6/00

(52) U.S. Cl. 600/475; 600/473; 600/559

(57) ABSTRACT

To enable the user to see if the field of view of an infrared sensor faces an eardrum direction. A biological information collecting device (100) includes: an infrared sensor (108) for sensing the infrared radiation emitted from inside an acoustic foramen (200); an acoustic wave output section (152) that is arranged so as to emit an acoustic wave toward the field of view (F) of the infrared sensor (108); and a computing section (110) for deriving biological information based on the output of the infrared sensor (108).

This is a continuation of International Application No. PCT/JP2008/050861, with an international filing date of Jan. 23, 2008, which claims priority of Japanese Patent Application No. 2007-013313, filed on Jan. 24, 2007, the contents of which are hereby incorporated by reference.

### BACKGROUND OF THE INVENTION

1. Field of the Invention

The present invention relates to a biological information collecting device for collecting biological information non-invasively using infrared radiation emitted by an eardrum and also relates to a method for controlling such a device.

2. Description of the Related Art

Various non-invasive blood glucose level meters for calculating the glucose level by measuring the intensity of the radiation emitted by an eardrum have been proposed as conventional biological information collecting devices. For example, PCT International Application Japanese National-Phase Publication No. 05-506171 discloses a non-invasive blood glucose level meter, which includes a mirror that is small enough to be inserted into the ear canal and which irradiates the eardrum with a near-infrared radiation or a thermal ray, and detects the ray reflected from the eardrum, by way of the mirror, thereby calculating the glucose level based on the result of the detection. On the other hand, PCT International Application Japanese National-Phase Publication No. 2002-513604 discloses a non-invasive blood glucose level meter, which includes a probe to be inserted into the acoustic foramen and which detects an infrared radiation produced from the internal ear and emitted through the eardrum using the probe with the eardrum and the ear canal cooled and subjects the infrared radiation detected to a spectral analysis, thereby obtaining the glucose level. Furthermore, PCT International Application Japanese National-Phase Publication No. 2001-503999 discloses a non-invasive blood glucose level meter, which includes a reflective mirror to be inserted into the acoustic foramen and which detects the radiation emitted by the eardrum by way of the reflective mirror and subjects the radiation detected to a spectral analysis, thereby obtaining the glucose level.

With these conventional biological information collecting devices, however, the user cannot see which direction the mirror or probe inserted into the acoustic foramen now faces, and it is difficult to set the measuring point right on the eardrum accurately inside the acoustic foramen. Consequently, the result of measurements should contain too many errors to guarantee high reliability.

In order to overcome the problems described above, an object of the present invention is to provide a technique for enabling the user to see if the field of view of the infrared sensor now faces the eardrum direction.

### SUMMARY OF THE INVENTION

A biological information collecting device according to the present invention includes: an infrared sensor for sensing an infrared radiation that has been emitted from inside an acoustic foramen; an acoustic wave output section, which is arranged so as to emit an acoustic wave toward the field of view of the infrared sensor; and a computing section for deriving biological information based on the output of the infrared sensor.

The acoustic wave output section may include a sound source that emits the acoustic wave and an acoustic waveguide for guiding the emitted acoustic wave through the acoustic foramen and toward the field of view of the infrared sensor.

In such a preferred embodiment, the acoustic wave that has been emitted from the sound source can be pointed toward the acoustic foramen with good directivity. In this case, if the device is designed such that the direction in which the acoustic wave is guided through the acoustic waveguide falls into the field of view of the infrared sensor, then the acoustic wave can be pointed toward the same direction as the one that the field of view of the infrared sensor faces. As a result, it can be determined more accurately whether or not the field of view of the infrared sensor faces the eardrum direction.

The biological information collecting device may further include an acoustic wave detector for detecting a reflected wave that has been produced by the reflection of the acoustic wave from inside the acoustic foramen, and a decision section for determining, based on a result of detection done by the acoustic wave detector, whether or not an eardrum falls into the field of view of the infrared sensor.

Then it is determined automatically whether or not the field of view of the infrared sensor faces the eardrum direction. Thus, there is no need for the user to determine whether or not the field of view of the infrared sensor faces the eardrum direction by him- or herself.

The biological information collecting device may further include an acoustic waveguide for guiding the reflected wave from inside the acoustic foramen toward the acoustic wave detector.

In that case, among various reflected waves produced inside the acoustic foramen, only the reflected wave that has been directed toward the acoustic waveguide can be sorted out. As a result, it can be determined more accurately, based on the intensity of the reflected wave that has been detected by the acoustic wave detector, whether or not the field of view of the infrared sensor faces the eardrum direction inside the acoustic foramen.

The biological information collecting device may further include a comparison section for comparing the intensity of the reflected wave, which has been detected by the acoustic wave detector, to a predetermined threshold value. With a result of comparison made by the comparison section also taken into consideration, the decision section may determine whether or not the eardrum falls into the field of view of the infrared sensor.

The biological information collecting device may further include a threshold value storage section for storing a predetermined threshold value. The predetermined threshold value has been determined in advance based on the intensity of the reflected wave. And the comparison section may compare the intensity of the reflected wave that has been detected by the acoustic wave detector to the predetermined threshold value.

If the field of view of the infrared sensor faces the eardrum direction, the reflected wave to be detected by the acoustic wave detector will have a decreased intensity. On the other hand, if the field of view of the infrared sensor faces the ear canal, the ratio of the intensity of the acoustic wave to that of the reflected wave detected by the acoustic wave detector will increase.

That is why by setting the threshold value somewhere between the intensity of the reflected wave to be detected by the acoustic wave detector in a situation where the field of view of the infrared sensor faces the eardrum direction and that of the reflected wave to be detected by the acoustic wave detector in a situation where the field of view of the infrared sensor faces the ear canal, it can be determined that the field of view of the infrared sensor faces the eardrum direction if the intensity of the reflected wave detected by the acoustic wave detector is found to be less than the threshold value as a result of the comparison made by the comparison section. On the other hand, if the intensity of the reflected wave detected by the acoustic wave detector is equal to or greater than the threshold value, then it can be determined that the field of view of the infrared sensor does not face the eardrum direction.

The biological information collecting device may further include an alarm output section for outputting an alarm based on a result of comparison made by the comparison section.

In such a preferred embodiment, if the alarm output section is designed so as to output an alarm in a situation where it has been determined, based on a result of comparison made by the comparison section, that the field of view of the infrared sensor does not face the eardrum direction, the user can be notified that the field of view of the infrared sensor is not directed appropriately.

The acoustic wave output section may emit the acoustic wave with at least one frequency selected from the frequency range of 1,000 Hz through 6,000 Hz.

The acoustic wave output section may emit the acoustic wave as a simple tone. Alternatively, the acoustic wave output section may emit the acoustic wave with a constant intensity. In that case, the acoustic wave emitted toward the acoustic foramen will have no intensity variation, and therefore, the user can sense a variation in loudness more easily.

Still alternatively, the acoustic wave output section may emit the acoustic wave with a constant frequency. In that case, there will be no variation in sound tone, and therefore, the user can sense a variation in loudness more easily.

The acoustic wave output section may emit first and second acoustic waves to be reflected from an eardrum with mutually different reflectances. The acoustic wave detector may sense at least one of the reflected waves of the first and second acoustic waves. And the computing section may derive the biological information based on the output of the infrared sensor when the acoustic wave detector has detected the reflected wave.

By setting the respective frequencies of the first and second acoustic waves such that the reflectance of the first acoustic wave from the eardrum becomes higher than that of the second acoustic wave from the eardrum, if the field of view of the infrared sensor faces the eardrum direction, the ratio of the intensity of the first acoustic wave to that of the reflected one thereof to be detected by the acoustic wave detector will become larger than the ratio of the intensity of the second acoustic wave to that of the reflected one thereof to be detected by the acoustic wave detector.

On the other hand, if the field of view of the infrared sensor faces the ear canal, then the reflectance from the ear canal will be high in both of the two acoustic waves. Consequently, the ratio of the intensity of the first acoustic wave to that of the reflected one thereof and the ratio of the intensity of the second acoustic wave to that of the reflected one thereof to be detected by the acoustic wave detector will be both high.

Furthermore, if the field of view of the infrared sensor faces neither the eardrum direction nor the ear canal direction or if the biological information collecting device has been inserted into the acoustic foramen insufficiently, then portions of the reflected ones of the first and second acoustic waves that reach the inserting portion will decrease. As a result, the ratio of the intensity of the first acoustic wave to that of the reflected one thereof to be detected by the acoustic wave detector and the ratio of the intensity of the second acoustic wave to that of the reflected one thereof to be detected by the acoustic wave detector will be both small.

Consequently, the biological information collecting device of the present invention can determine, based on the respective intensities of the reflected ones of the first and second acoustic waves to be detected by the acoustic wave detector, whether or not the field of view of the infrared sensor faces the eardrum direction.

The biological information collecting device may further include an acoustic wave detector for detecting a reflected wave that has been produced by the reflection of the acoustic wave from inside the acoustic foramen, and a decision section for determining, based on a result of detection done by the acoustic wave detector, whether or not an eardrum falls into the field of view of the infrared sensor. The decision section may determine, based on the intensities of the reflected waves of the first and second acoustic waves, whether or not the eardrum falls into the field of view of the infrared sensor.

The acoustic wave output section may include: a sound source having the ability to emit one of the first and second acoustic waves selectively; a first acoustic waveguide for guiding the first and second acoustic waves, emitted from the sound source, through the acoustic foramen and toward the field of view of the infrared sensor; and a second acoustic waveguide for guiding the reflected waves of the first and second acoustic waves from inside the acoustic foramen toward the acoustic wave detector.

Then the first and second acoustic waves emitted from the sound source can be pointed, with good directivity, toward the direction that the field of view of the infrared sensor faces inside the acoustic foramen. Also, among various reflected waves produced inside the acoustic foramen, only a part of the reflected wave that has been directed toward the second acoustic waveguide can be sorted out. As a result, it is possible to determine more accurately, based on the intensities of reflected ones of the first and second acoustic waves to be detected by the acoustic wave detector, whether or not the field of view of the infrared sensor faces the eardrum direction inside the acoustic foramen.

The biological information collecting device may further include a comparison section for comparing the respective intensities of the reflected waves of the first and second acoustic waves, which have been detected by the acoustic wave detector, to at least one threshold value. With a result of comparison made by the comparison section also taken into consideration, the decision section may determine whether or not the eardrum falls into the field of view of the infrared sensor.

The biological information collecting device may further include a threshold value storage section for storing the at least one threshold value. The at least one threshold value may include first and second threshold values. And the comparison section may compare the respective intensities of the reflected waves of the first and second acoustic waves that have been detected by the acoustic wave detector to the first and second threshold values, respectively.

In this case, the threshold value preferably includes a first threshold value for the intensity of the reflected wave of the first acoustic wave and a second threshold value for that of the reflected wave of the second acoustic wave. The device preferably further includes a threshold value storage section that stores the first and second threshold values. And the comparison section preferably compares the intensity of the reflected wave of the first acoustic wave that has been detected by the acoustic wave detector to the first threshold value and also compares the intensity of the reflected wave of the second acoustic wave that has been detected by the acoustic wave detector to the second threshold value.

If the field of view of the infrared sensor faces the eardrum direction, the ratio of the intensity of the first acoustic wave to that of the reflected one thereof to be detected by the acoustic wave detector will become larger than the ratio of the intensity of the second acoustic wave to that of the reflected one thereof to be detected by the acoustic wave detector. On the other hand, if the field of view of the infrared sensor faces the ear canal, then the ratio of the intensity of the first acoustic wave to that of the reflected one thereof and the ratio of the intensity of the second acoustic wave to that of the reflected one thereof to be detected by the acoustic wave detector will be both high. Furthermore, if the field of view of the infrared sensor faces neither the eardrum direction nor the ear canal direction, then the ratio of the intensity of the first acoustic wave to that of the reflected one thereof to be detected by the acoustic wave detector and the ratio of the intensity of the second acoustic wave to that of the reflected one thereof to be detected by the acoustic wave detector will be both small.

That is why the first threshold value is set somewhere between the intensity of the reflected one of the first acoustic wave to be detected by the acoustic wave detector when the field of view of the infrared sensor faces the eardrum direction and that of the reflected one of the first acoustic wave to be detected by the acoustic wave detector when the field of view of the infrared sensor faces neither the eardrum direction nor the ear canal direction. Also, the second threshold value is set somewhere between the intensity of the reflected one of the second acoustic wave to be detected by the acoustic wave detector when the field of view of the infrared sensor faces the ear canal direction and that of the reflected one of the second acoustic wave to be detected by the acoustic wave detector when the field of view of the infrared sensor faces the eardrum direction. As a result, if a result of comparison made by the comparison section reveals that the intensity of the reflected one of the first acoustic wave detected by the acoustic wave detector is greater than the first threshold value and that the intensity of the reflected one of the second acoustic wave detected by the acoustic wave detector is less than the second threshold value, then it can be determined that the field of view of the infrared sensor faces the eardrum direction. On the other hand, if the intensity of the reflected one of the first acoustic wave detected by the acoustic wave detector is equal to or smaller than the first threshold value or if the intensity of the reflected one of the second acoustic wave detected by the acoustic wave detector is equal to or greater than the second threshold value, then it can be determined that the inserting portion that has been introduced into the acoustic foramen does not face the eardrum.

Alternatively, the threshold value may be set with respect to a differential value between the respective intensities of reflected ones of the first and second acoustic waves. The device may further include a threshold value storage section that stores the threshold value. And the comparison section may compare the differential value between respective intensities of reflected ones of the first and second acoustic waves that have been detected by the acoustic wave detector to the threshold value stored in the threshold value storage section.

If the field of view of the infrared sensor faces the eardrum direction, the ratio of the intensity of the first acoustic wave to that of the reflected one thereof to be detected by the acoustic wave detector will become larger than the ratio of the intensity of the second acoustic wave to that of the reflected one thereof to be detected by the acoustic wave detector. On the other hand, if the field of view of the infrared sensor faces the ear canal direction, then the ratio of the intensity of the first acoustic wave to that of the reflected one thereof and the ratio of the intensity of the second acoustic wave to that of the reflected one thereof to be detected by the acoustic wave detector will be both high. Furthermore, if the field of view of the infrared sensor faces neither the eardrum direction nor the ear canal direction or if the biological information collecting device has been inserted into the acoustic foramen insufficiently, then the ratio of the intensity of the first acoustic wave to that of the reflected one thereof to be detected by the acoustic wave detector and the ratio of the intensity of the second acoustic wave to that of the reflected one thereof to be detected by the acoustic wave detector will be both small.

That is why the differential value between the respective intensities of reflected ones of the first and second acoustic waves to be detected by the acoustic wave detector becomes greater when the field of view of the infrared sensor faces the eardrum direction than not.

That is why the threshold value to be stored in the threshold value storage section is set somewhere between the two differential values representing respective differences between the intensities of reflected ones of the first and second acoustic waves to be detected by the acoustic wave detector when the field of view of the infrared sensor faces the eardrum direction and when the field of view of the infrared sensor does not face the eardrum direction, respectively. As a result, if a result of comparison made by the comparison section reveals that the differential value between the respective intensities of reflected ones of the first and second acoustic waves detected by the acoustic wave detector is greater than the threshold value, then it can be determined that the field of view of the infrared sensor faces the eardrum direction. On the other hand, if the differential value between the respective intensities of reflected ones of the first and second acoustic waves detected by the acoustic wave detector is equal to or smaller than the threshold value, then it can be determined that the inserting portion that has been introduced into the acoustic foramen does not face the eardrum direction.

The biological information collecting device may further include a threshold value storage section for storing the at least one threshold value. A differential value, showing a difference between the respective intensities of the reflected waves of the first and second acoustic waves, which have been detected by the acoustic wave detector, may be compared to the at least one threshold value.

The biological information collecting device may further include an alarm output section for outputting an alarm based on a result of comparison made by the comparison section.

The acoustic wave output section may emit the first acoustic wave with at least one frequency selected from the frequency range of 20 Hz through 800 Hz and may also emit the second acoustic wave with at least one frequency selected from the frequency range of 1,000 Hz through 6,000 Hz. Then the reflectance of the first acoustic wave from the eardrum will be higher than that of the second acoustic wave from the eardrum.

The acoustic wave output section may emit the first and second acoustic waves as simple tones.

Alternatively, the acoustic wave output section may also emit the first and second acoustic waves, each of which has a constant intensity.

Still alternatively, the acoustic wave output section may also emit the first and second acoustic waves, each of which has a constant frequency.

The biological information collecting device may further include a spectral element for subjecting the infrared radiation, which has been emitted from the acoustic foramen, to some spectral process.

The biological information collecting device may further include a storage section for storing an output signal value of the infrared sensor in association with a result of decision made by the decision section.

A method according to the present invention is a method for controlling the biological information collecting device described above. The device further includes a control section for controlling the infrared sensor, the acoustic wave output section, the acoustic wave detector, the computing section, the decision section and the storage section. The method includes the steps of: (a) getting the infrared radiation, emitted from inside the acoustic foramen, sensed by the infrared sensor; (b) getting the first and second acoustic waves sequentially emitted from the acoustic wave output section; (c) getting the reflected waves of the first and second acoustic waves detected by the acoustic wave detector; (d) making the decision section determine, based on the respective intensities of the reflected waves of the first and second acoustic waves that have been detected by the acoustic wave detector, whether or not the eardrum falls into the field of view of the infrared sensor; (e) storing the output signal values of the infrared sensor in the storage section in association with a result of decision made by the decision section; and (f) getting an output signal value when it is determined by the decision section that the eardrum falls into the field of view of the infrared sensor read by the computing section from the output signal values stored in the output signal storage section and getting the biological information derived by the computing section based on the output signal value read.

According to this scheme, the computing section can automatically extract the output signal of the infrared sensor when the field of view of the infrared sensor faces the eardrum direction, and therefore, can derive the biological information based on the intensity of the infrared radiation that is suitable for measurements. As a result, the biological information can be collected even more accurately.

Another method according to the present invention is a method for controlling the biological information collecting device described above. The device further includes a control section for controlling the infrared sensor, the acoustic wave output section, the acoustic wave detector, and the decision section. The method includes the steps of: (a) getting the first and second acoustic waves sequentially emitted from the acoustic wave output section; (b) getting the reflected waves of the first and second acoustic waves detected by the acoustic wave detector; (c) making the decision section determine, based on the respective intensities of the reflected waves of the first and second acoustic waves that have been detected by the acoustic wave detector, whether or not the eardrum falls into the field of view of the infrared sensor; and if it has been determined in the step (c) that the eardrum falls into the field of view of the infrared sensor, (d) getting sensing of the infrared radiation, emitted from inside the acoustic foramen, started by the infrared sensor.

According to this scheme, sensing of the infrared radiation that has been emitted from inside the acoustic foramen gets started by automatically sensing the field of view of the infrared sensor turned to the eardrum direction that is suitable for measurements. As a result, the biological information can be collected even more accurately.

The biological information collecting device of the present invention may further include a correlation data storage section for storing correlation data representing a correlation between the output signal of the infrared sensor and the biological information, a display section for presenting the biological information that has been derived by the computing section, and a power supply for supplying power to operate the biological information collecting device.

The computing section may convert the output signal of the infrared sensor into biological information by reading the correlation data from the correlation data storage section and referring to that data.

The correlation data representing a correlation between the output signal of the infrared sensor and the biological information may be acquired by monitoring the output signal of the infrared sensor for a patient with known biological information (such as a blood glucose level) and analyzing the correlation between the output signal of the infrared sensor and the biological information.

According to the present invention, it can be determined whether or not the field of view of the infrared sensor faces the eardrum direction. As a result, the measuring spot can be set right on the eardrum and highly accurate biological information can be collected using the infrared radiation that has been emitted from the eardrum.

Other features, elements, processes, steps, characteristics and advantages of the present invention will become more apparent from the following detailed description of preferred embodiments of the present invention with reference to the attached drawings.

### BRIEF DESCRIPTION OF DRAWINGS

FIG. 1 is a perspective view illustrating the appearance of a biological information collecting device as a first preferred embodiment of the present invention.

FIG. 2 illustrates the configuration of the biological information collecting device of the first preferred embodiment.

FIG. 3 is a perspective view illustrating the optical filter wheel of the biological information collecting device.

FIG. 4 shows the frequency characteristic of the reflectance of an acoustic wave from an eardrum.

FIG. 5 is a perspective view illustrating the appearance of a biological information collecting device as a second preferred embodiment of the present invention.

FIG. 6 illustrates the configuration of the biological information collecting device of the second preferred embodiment.

FIG. 7 illustrates the configuration of a biological information collecting device as a third preferred embodiment of the present invention.

FIG. 8 is a perspective view illustrating the appearance of a biological information collecting system as a sixth preferred embodiment of the present invention.

FIG. 9 illustrates the respective internal configurations of the measuring section and the body section of the biological information collecting system.

FIG. 10 is a partially cutaway cross-sectional view illustrating the internal configuration of the measuring section.

FIG. 11 is a cross-sectional view thereof as viewed on the plane A-A shown in FIG. 10.

FIG. 12 is a cross-sectional view thereof as viewed on the plane B-B shown in FIG. 10.

FIGS. 13(a) through 13(d) are plan views illustrating an example of a cam gear portion as viewed from a cam portion.

FIG. 14 illustrates the configuration of a biological information collecting device as a seventh preferred embodiment of the present invention.

### DETAILED DESCRIPTION OF PREFERRED EMBODIMENTS

In the following description, first, it will be described how to determine in principle whether or not the field of view of an infrared sensor faces the eardrum direction. After that, it will be described how to collect biological information using the infrared radiation that has been emitted from the eardrum. And then various preferred embodiments of the present invention that are designed to determine whether or not the field of view of the infrared sensor faces the eardrum direction will be set forth in detail.

As used herein, the “biological information” is defined to be pieces of information that represent the physical health condition of an organism. Examples of biological information according to the present invention include a glucose concentration (i.e., blood glucose level), a hemoglobin concentration, a cholesterol concentration, a neutral fat concentration, a protein concentration, the concentrations of other chemical constituents included in an organism and body temperature.

A biological information collecting device according to the present invention includes: an infrared sensor for sensing an infrared radiation that has been emitted from inside an acoustic foramen; an acoustic wave output section, which is arranged so as to emit an acoustic wave toward the field of view of the infrared sensor; and a computing section for deriving biological information based on the output of the infrared sensor.

As used herein, the “infrared radiation emitted from inside the acoustic foramen” includes an infrared radiation emitted from inside the acoustic foramen as a thermal radiation from a vital tissue itself such as the eardrum or the ear canal inside the acoustic foramen and an infrared radiation emitted from inside the acoustic foramen as the infrared radiation that has been incident onto the acoustic foramen and then reflected from the vital tissue inside the acoustic foramen.

The acoustic wave that has been emitted toward the acoustic foramen vibrates the eardrum. And the vibration of the eardrum is transmitted through the auditory ossicle to the cochlea and converted into an electrical signal there. Then the electrical signal generated by the conversion is transmitted to the brain through acoustic nerves. As a result, the user senses it as sound. However, the acoustic wave that has been emitted toward the acoustic foramen is reflected to a certain degree according to the acoustic impedance of the vital tissue such as the ear canal or the eardrum.

The acoustic impedance changes according to the frequency of the acoustic wave. Generally speaking, a vital tissue has high acoustic impedance and reflects the acoustic wave well. The ear canal also has so high acoustic impedance as to reflect the acoustic wave well.

That is why if the acoustic wave has been emitted toward the ear canal, the acoustic wave is reflected from the ear canal so much that the acoustic wave transmitted to the eardrum has low intensity. As a result, the user senses the acoustic wave as a faint sound. On the other hand, if the acoustic wave has been emitted toward the eardrum, the user senses the acoustic wave as a loud sound.

If the direction in which the acoustic wave is emitted from the sound source falls into the field of view of the infrared sensor, then the user can see that the field of view of the infrared sensor faces the eardrum direction when he or she hears the acoustic wave as a loud sound. Consequently, the biological information collecting device of the present invention can determine whether or not the field of view of the infrared sensor faces the eardrum direction.

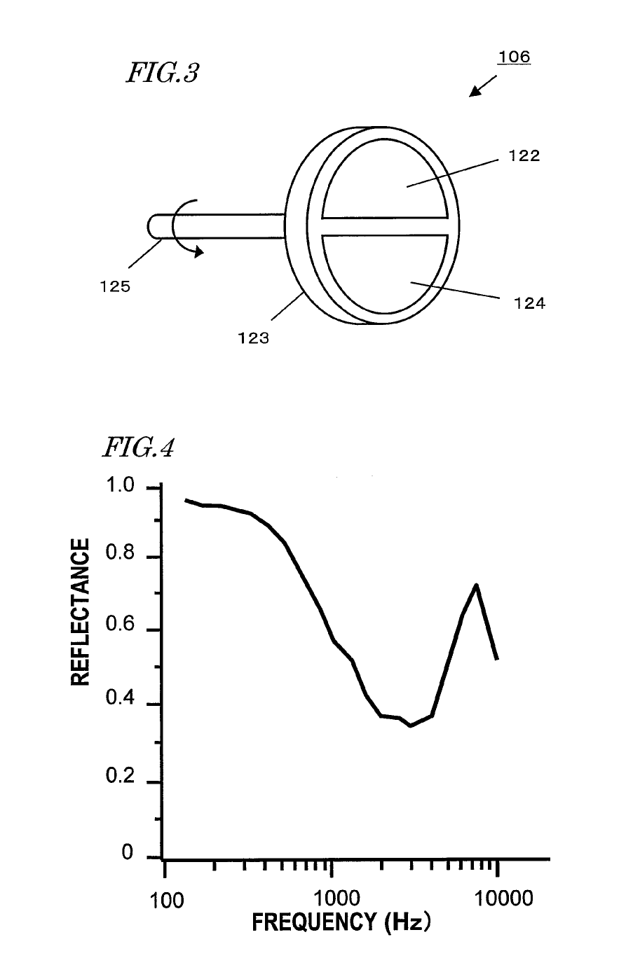

It is known that the acoustic impedance of the eardrum changes significantly in the audible range of a human being. FIG. 4 shows the relation between the power reflectance of the acoustic wave from the eardrum and the frequency thereof. As shown in FIG. 4, in a low frequency range of 20 Hz to 800 Hz, for example, the eardrum has such high acoustic impedance as to reflect well acoustic waves falling within the frequency range of 20 Hz to 800 Hz. On the other hand, as for acoustic waves falling within the frequency range of 1,000 Hz to 6,000 Hz, the reflectance from the eardrum is low. This is a frequency range in which the acoustic wave is transmitted to the internal ear so much as to be audible easily for a human being. That is why the acoustic wave preferably has a frequency falling within the range of 1,000 Hz to 6,000 Hz.

In the latter case, since this is a frequency range in which the incoming acoustic wave is easily audible for a human being, the user can determine more easily, by the sound he or she has heard, whether or not the field of view of the infrared sensor faces the eardrum.

If the infrared radiation emitted by an organism is measured, information about a biological constituent concentration such as a blood glucose level can be obtained. Hereinafter, that principle will be illustrated first, and then the functional block arrangement of a biological information collecting device according to the present invention, operating on that principle, will be described. After that, specific preferred embodiments of a biological information collecting device according to the present invention will be set forth.

The radiation energy W of the infrared radiation that has been emitted as thermal radiation from an organism is represented by the following Equations (1) and (2):

$$\begin{matrix}W = S {\int }_{{λ}_{1}}^{{λ}_{2}} ɛ ( λ ) \cdot {W}_{0} ( T , λ ) d λ ( W ) & ( 1 ) \\ {W}_{0} ( λ , T ) = 2 {hc}^{2} {\{ {λ}^{5} \cdot [ exp ( hc / λ kT ) - 1 ] \}}^{- 1} ( W / {cm}^{2} \cdot μ m ) & ( 2 )\end{matrix}$$

where W is the radiation energy of the infrared radiation that has been emitted as thermal radiation from an organism, ε (λ) is the emissivity of the organism at a wavelength λ, W₀ (λ, T) is the spectral radiant density of a thermal radiation from blackbody at the wavelength λ and a temperature T, h is Planck's constant (where h=6.625×10⁻³⁴ W·S²)), c is the velocity of light (where c=2.998×10¹⁰ cm/s), λ₁ and λ₂ are wavelengths (μm) of infrared radiations emitted from the organism, T is the temperature (K) of the organism, S is the detection area (cm²) and k is Boltzmann constant.

According to Equation (1), if the detection area S is constant, the radiation energy W of the infrared radiation emitted as a thermal radiation from an organism depends on the emissivity ε (λ) of the organism at a wavelength λ. According to the Kirchhoff's law on radiation, the emissivity and the absorptivity are equal to each other at the same temperature and at the same wavelength.

ε(λ)=α(λ) (3)

where α (λ) is the absorptivity of the organism at the wavelength λ.

That is why it can be seen that when the emissivity needs to be obtained, the absorptivity may be calculated. Based on the principle of energy conservation, the absorptivity, the transmittance and the reflectance satisfy the following Equation (4):

α(λ)+r(λ)+t(λ)=1 (4)

where r (λ) is the reflectance of the organism at the wavelength λ and t (λ) is the transmittance of the organism at the wavelength λ.

Therefore, the emissivity can be calculated by the following Equation (5) using the transmittance and the reflectance:

ε(λ)=α(λ)=1−r(λ)−t(λ) (5)

The transmittance is represented as the ratio of the intensity of the light that has been transmitted through an object of interest to that of the incoming light. The intensity of the incoming light and that of the light that has been transmitted through the object of interest are given by the Lambert-Beer law:

$${I}_{t} ( λ ) = {I}_{0} ( λ ) exp ( - \frac{4 π k ( λ )}{λ} d ) \qquad ( 6 )$$

where Iₜ is the intensity of the transmitted light, I₀ is the intensity of the incoming light, d is the thickness of the organism and k (λ) is the extinction coefficient of the organism at the wavelength λ. The extinction coefficient of the organism represents absorption of the light into the organism.

Consequently, the transmittance is given by the following Equation (7):

$$t ( λ ) = exp ( - \frac{4 π k ( λ )}{λ} d ) \qquad ( 7 )$$

Next, the reflectance will be described. The reflectance should be calculated as the average of reflectances in all directions. In this example, only the reflectance to perpendicularly incident light will be considered for the sake of simplicity. Supposing the refractive index of the air is one, the reflectance to the perpendicularly incident light is given by the following Equation (8):

$$r ( λ ) = \frac{{( n ( λ ) - 1 )}^{2} + {k}^{2} ( λ )}{{( n ( λ ) + 1 )}^{2} + {k}^{2} ( λ )} \qquad ( 8 )$$

where n (λ) is the refractive index of the organism at the wavelength λ.

Consequently, the emissivity is given by the following Equation (9):

$$\begin{matrix}ɛ ( λ ) = 1 - r ( λ ) - t ( λ ) \\ = 1 - \frac{{( n ( λ ) - 1 )}^{2} + {k ( λ )}^{2}}{{( n ( λ ) + 1 )}^{2} + {k ( λ )}^{2}} - exp ( - \frac{4 π k ( λ )}{λ} d )\end{matrix} \qquad ( 9 )$$

If the concentration of a constituent varies in an organism, the refractive index and the extinction coefficient of the organism will also change. The reflectance is usually as low as about 0.03 in the infrared range. Also, as can be seen from Equation (8), the reflectance does not depend on the refractive index or the extinction coefficient so much. That is why even if the refractive index and the extinction coefficient change due to a variation in biological constituent concentration, the reflectance will vary a little.

On the other hand, the transmittance heavily depends on the extinction coefficient as can be seen from Equation (7). For that reason, if the extinction coefficient of an organism (i.e., the degree of absorption of light into the organism) changes due to a variation in biological constituent concentration, the transmittance will change, too.

Thus, it can be seen that the radiation energy of the infrared radiation emitted as a thermal radiation from an organism depends on the concentration of the biological constituent. That is to say, the biological constituent concentration can be calculated based on the intensity of the radiation energy of the infrared radiation that has been emitted as a thermal radiation from the organism.

According to Equation (7), the transmittance depends on the thickness of the vital tissue. That is to say, the smaller the thickness of the vital tissue, the more significantly the transmittance will change with a variation in the extinction coefficient of the organism and the more easily the variation in biological constituent concentration can be detected. The eardrum has such a small thickness of about 60 μm to about 100 μm as to be suitable for determining the biological constituent concentration using infrared radiation.

Hereinafter, preferred embodiments of the present invention will be described with reference to the accompanying drawings.

### Embodiment 1

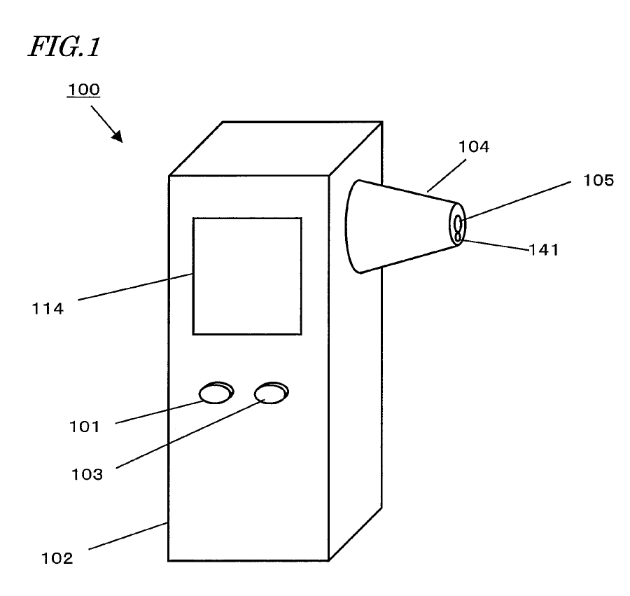

FIG. 1 is a perspective view illustrating the appearance of a biological information collecting device 100 as a first preferred embodiment of the present invention.

the Biological Information Collecting Device 100 includes a body 102 and an inserting portion 104 arranged on a side surface of the body 102. The body 102 includes a display 114 such as an LCD to show the biological constituent concentration measured, a power switch 101 to turn ON and OFF the biological information collecting device 100, and a measuring start switch 103 to start the measuring process. The inserting portion 104 includes an optical waveguide 105 for guiding the infrared radiation that has been emitted from the acoustic foramen into the biological information collecting device 100 and a first acoustic waveguide 141 for transmitting an acoustic wave from the body 102 into the acoustic foramen.

In this example, the opening of the first acoustic waveguide 141 is located at the end (or terminal) of the inserting portion 104. Thus, if the inserting portion 104 is inserted into the acoustic foramen so that the end of the inserting portion 104 faces the eardrum, then the opening of the first acoustic waveguide 141 also faces the eardrum.

Likewise, the opening of the optical waveguide 105 also faces the eardrum. That is why if the end of the inserting portion 104 faces the eardrum, the field of view of the infrared sensor is also directed toward the eardrum.

Therefore, this inserting portion 104 is designed such that the direction in which an acoustic wave is emitted through the opening of the first acoustic waveguide 141 falls into the field of view of the infrared sensor. The first acoustic waveguide 141 corresponds to the sound transmitting portion of the present invention. Anything may be used as the first acoustic waveguide 141 as long as it can guide an acoustic wave. For example, a hollow tube may be used.

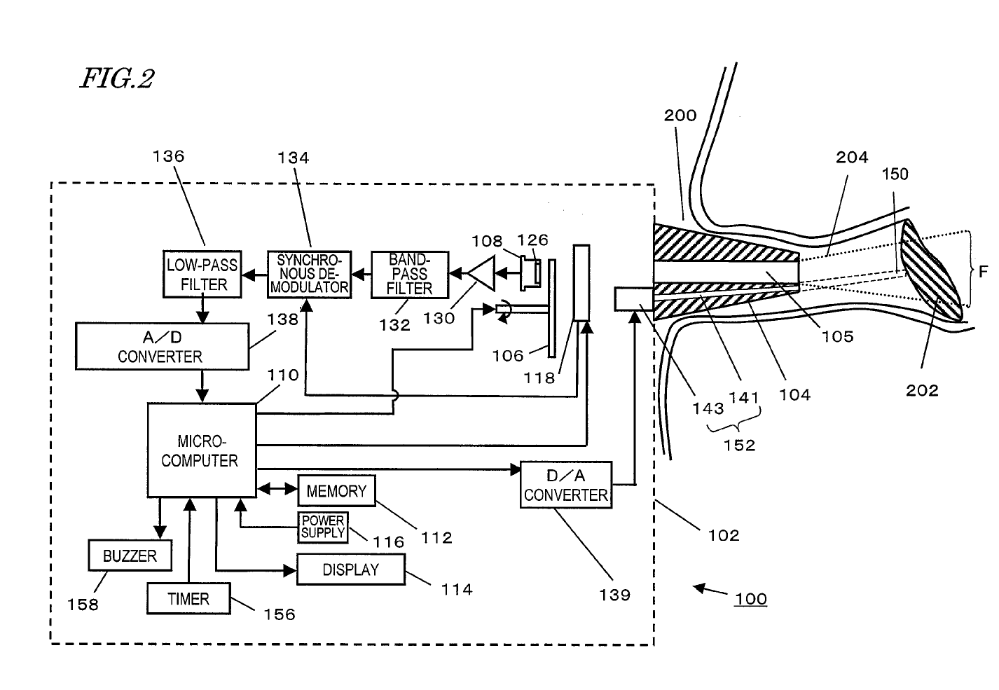

Next, the internal configuration of the body of the biological information collecting device 100 will be described with reference to FIGS. 2 and 3. FIG. 2 illustrates the configuration of the biological information collecting device 100 of the first preferred embodiment, and FIG. 3 is a perspective view illustrating the optical filter wheel 106 of the biological information collecting device 100 of the first preferred embodiment.

The body of the biological information collecting device 100 includes a chopper 118, an optical filter wheel 106, an infrared sensor 108, a pre-amplifier 130, a band-pass filter 132, a synchronous demodulator 134, a low-pass filter 136, an analog-to-digital (A/D) converter 138, a microcomputer 110, a memory 112, a display 114, a power supply 116, a timer 156, a sound source 143, a digital-to-analog (D/A) converter 139, and a buzzer 158. The microcomputer 110 may be implemented as a CPU (central processing unit), for example, and corresponds to the computing section of the present invention.

The power supply 116 supplies alternating current (AC) or direct current (DC) power to the microcomputer 110. A battery is preferably used as the power supply 116.

The sound source 143 has the function of producing an acoustic wave to beat on the acoustic foramen 200. In this preferred embodiment, the sound source 143 produces an acoustic wave as a simple tone with a single frequency of 1,200 Hz.

Any known sound source may be used without any particular restriction as long as the sound source can produce an acoustic wave. Examples of preferred sound sources include a loudspeaker to which an FM (Frequency Modulation) sound source is connected, a loudspeaker to which an MIDI (Musical Instrument Digital Interface) sound source is connected, and a buzzer or a piezoelectric element that produces an acoustic wave at a particular frequency.

The acoustic wave produced by the sound source 143 will be incident on the acoustic foramen 200 through the first acoustic waveguide 141. The acoustic wave that has been incident on the acoustic foramen 200 will be partially absorbed into, and partially reflected from, the eardrum 202, the ear canal 204 and other vital tissues.

In the description of this and other preferred embodiments of the present invention, the sound source 143 and the first acoustic waveguide 141 will be collectively referred to herein as an “acoustic wave output section 152”, which performs the function of emitting an acoustic wave toward the field of view of the infrared sensor 108.

As used herein, “to emit an acoustic wave toward the field of view of the infrared sensor 108” means emitting an acoustic wave from the sound source 143 through the first acoustic waveguide 141 such that the acoustic wave 150 reaches the field F, which is shown as the field of view of the infrared sensor 108. However, as shown in FIG. 1, the first acoustic waveguide 141 is not parallel to, but tilted with respect to, the optical waveguide 105. For that reason, the acoustic wave 150 just needs to fall within the field F within a gap of 1 cm to 2 cm, which is supposed to be left in a normal use between the open end of the optical waveguide 105 and the eardrum 202. It should be noted that the field F is defined with the reflection of the infrared radiation from the inner surface of the optical waveguide 105 taken into consideration and is broader than the opening of the optical waveguide 105 inside the acoustic foramen 200.

The chopper 118 chops the infrared radiation that has been emitted as a thermal radiation from the eardrum 202 and then guided into the body 102 through the optical waveguide 105, thereby transforming the infrared radiation into a high-frequency infrared signal. The operation of the chopper 118 is controlled in accordance with a control signal supplied from the microcomputer 110. The infrared radiation that has been chopped by the chopper 118 soon reaches the optical filter wheel 106.

In the optical filter wheel 106, first and second optical filters 122 and 124 are fitted into a ring 123 as shown in FIG. 3. In the example illustrated in FIG. 3, the first and second optical filters 122 and 124, both of which are semicircular, are fitted into the ring 123, thereby forming a disklike member. And at the center of that disklike member, arranged is a shaft 125.

By rotating the shaft 125 in the direction indicated by the arrow shown in FIG. 3, the optical filters to pass the infrared radiation that has been chopped by the chopper 118 may be switched from one of these two optical filters 122 and 124 into the other. The rotation of the shaft 125 is controlled in accordance with a control signal supplied from the microcomputer 110.

The shaft 125 preferably has its revolution synchronized with the rotation of the chopper 118 and is preferably controlled so as to turn 180 degrees while the chopper 118 is closed. This is because in that case, when the chopper 118 is opened next time, the infrared radiation to be chopped by the chopper 118 may be transmitted through the next optical filter. The optical filter wheel 106 corresponds to the spectral element of the present invention. Any spectral element may be used as long as the element can split an infrared radiation into multiple rays with mutually different wavelengths. For example, an optical filter, a spectral prism, a Michelson interferometer or a diffraction grating for transmitting an infrared radiation falling within a particular wavelength range may be used.

The infrared radiation that has been transmitted through the first or second optical filter 121 or 124 reaches the infrared sensor 108 with a sensing area 126. On reaching the infrared sensor 108, the infrared radiation is incident on the sensing area 126. The infrared sensor 108 receives the infrared radiation and transforms the infrared radiation into an electrical signal representing its intensity.

Any sensor may be used as the infrared sensor 108 as long as the sensor can detect radiations having wavelengths falling within the infrared range of the spectrum. For example, the infrared sensor 108 may be a pyroelectric sensor, a thermopile, a bolometer, an HgCdTe (MCT) detector or a Golay cell. Optionally, multiple infrared sensors may be provided.

The electrical signal is output from the infrared sensor 108 to the pre-amplifier 130 and then amplified there. Then, the amplified electrical signal has its signal components filtered out by the band-pass filter 132 except those falling within a frequency range, of which the center frequency is defined by the chopping frequency. As a result, noise caused by some statistical fluctuation such as thermal noise can be minimized.

The electrical signal that has been subjected to the filtering process by the band-pass filter 132 is synchronized with the chopping frequency of the chopper 118 and integrated by the synchronous demodulator 134 so as to be demodulated into a DC signal.

Next, the electrical signal that has been demodulated by the synchronous demodulator 134 has its high frequency components filtered out by the low-pass filter 136. In this manner, its noise can be further reduced.

Subsequently, the electrical signal that has been subjected to the filtering process by the low-pass filter 136 is converted by the A/D converter 138 into a digital signal, which is then input to the microcomputer 110. In this case, the electrical signal that has come from any of the optical filters by way of the infrared sensor 108 can have its source identified (i.e., it is possible to determine which of those optical filters the infrared radiation, represented by the electrical signal, has been transmitted through) by using a control signal for the shaft 125 as a trigger. The duration of an electrical signal associated with the same optical filter is defined as an interval after the microcomputer 110 has output a control signal for the shaft 125 and before it outputs the next shaft control signal. By calculating the integral of the electrical signals associated with the respective optical filters on the memory 112 and then working out its average, the noise can be further reduced. That is why the measured values are preferably integrated.

In the memory 112, stored is correlation data that shows a correlation between the signal values of the electrical signals corresponding to the respective intensities of the infrared radiations transmitted through the first and second optical filters 122 and 124 and the biological constituent concentration. The microcomputer 110 reads this concentration correlation data from the memory 112, calculates a digital signal per unit time based on the digital signal that has been stored in the memory 112 by reference to the concentration correlation data, and converts the digital signal into a biological constituent concentration. The memory 112 corresponds to the correlation data storage section of the present invention, as which a memory such as a RAM or a ROM may be used.

Then, the biological constituent concentration that has been worked out by the microcomputer 110 is output to and presented on the display 114. The display 114 corresponds to the display section of the present invention.

The first optical filter 122 has such a spectral characteristic as to transmit infrared radiation that falls within a wavelength range including the wavelength to be absorbed into the biological constituent under measurement (which will be referred to herein as “measuring wavelength range”). On the other hand, the second optical filter 124 has a different spectral characteristic from the first optical filter's 122. Specifically, the second optical filter 124 has such a spectral characteristic as to transmit infrared radiation that falls within a wavelength range including a wavelength to be absorbed into not the biological constituent under measurement but another biological constituent that would interfere with the measurement of the target biological constituent (which will be referred to herein as “reference wavelength range”). In this case, that another biological constituent may be any constituent that is included a lot in the organism other than the biological constituent under measurement.

For example, glucose has an infrared absorption spectrum with a peak of absorption around 9.6 μm. That is why if the biological constituent under measurement is glucose, the first optical filter 122 preferably has such a spectral characteristic as to transmit infrared radiation that falls within a wavelength range including 9.6 μm.

Meanwhile, protein, included a lot in an organism, would absorb infrared radiation around 8.5 μm, while glucose would not absorb infrared radiation around that wavelength. That is why the second optical filter 124 preferably has such a spectral characteristic as to transmit infrared radiation that falls within a wavelength range including 8.5 μm.

The correlation data stored in the memory 112 to show the correlation between the respective signal values of the electrical signals representing the intensities of the infrared radiations that have been transmitted through the first and second optical filters 122 and 124 and the biological constituent concentration may be acquired in the following manner, for example.

First, as for a patient with a known biological constituent concentration such as a blood glucose level, the infrared radiation that has been emitted from his or her eardrum 202 has its intensity measured. In this case, electrical signals representing the intensities of infrared radiations falling within the wavelength ranges to be transmitted by the first and second optical filters 122 and 124 are obtained. By making such measurement on a number of patients with mutually different biological constituent concentrations, multiple sets of data, each including the electrical signals representing the intensities of infrared radiations falling within the wavelength ranges to be transmitted by the first and second optical filters 122 and their associated biological constituent concentrations, can be collected.

Next, by analyzing these data sets that have been collected in this manner, correlation data is obtained. For example, a multivariate analysis is carried out by either a multiple regression analysis such as partial least squares regression (PLS) method or a neural network method on the electrical signals representing the intensities of infrared radiations falling within the wavelength ranges to be transmitted by the first and second optical filters 122 and their associated biological constituent concentrations. As a result, a function showing a correlation between the electrical signals representing the intensities of infrared radiations falling within the wavelength ranges to be transmitted by the first and second optical filters 122 and 124 and their associated biological constituent concentrations can be obtained.

Also, the first optical filter 122 may have such a spectral characteristic as to transmit infrared radiation falling within a measuring wavelength range and the second optical filter 124 may have such a spectral characteristic as to transmit infrared radiation falling within a reference wavelength range. In that case, the difference between the signal values of the electrical signals representing the intensities of infrared radiations falling within the wavelength ranges to be transmitted by the first and second optical filters 122 and 142 may be calculated, and the correlation between that difference and its associated biological constituent concentration may be obtained as the correlation data by performing a linear regression analysis such as a minimum square method, for example.

Hereinafter, it will be described with reference to FIGS. 1, 2 and 3 how the biological information collecting device of this preferred embodiment operates.

First, when the user presses the power switch 101 of the biological information collecting device 100, the power is turned ON inside the body 102 to get the biological information collecting device 100 ready to make measurements.

Next, the user holds the body 102 in his or her hand to introduce the inserting portion 104 into his or her ear canal 204 as shown in FIG. 2. In this case, the inserting portion 104 is introduced such that the tip of the optical waveguide 105 is directed toward the eardrum 202. The inserting portion 104 is a conical hollow tube that increases its diameter from the tip of the inserting portion 104 toward the portion connected to the body 102. That is why the inserting portion 104 has such a structure as to prevent itself from being inserted any deeper than the position where the outside diameter of the inserting portion 104 gets equal to the inside diameter of the acoustic foramen 200.

Subsequently, when the user presses the measuring start switch 103 of the biological information collecting device 100 with the biological information collecting device 100 held at the position where the outside diameter of the inserting portion 104 gets equal to the inside diameter of the acoustic foramen 200, the microcomputer 110 activates the sound source 143, thereby producing an acoustic wave from the sound source 143. In this example, the sound source 143 produces an acoustic wave as a simple tone with a single frequency of 1,200 Hz at a constant intensity.

The acoustic wave that has been produced by the sound source 143 is transmitted through the first acoustic waveguide 141 and into the acoustic foramen 200.

Then, the acoustic wave propagating through the acoustic foramen 200 is partially absorbed into, and partially reflected from, the eardrum 202, the ear canal 204 and other vital tissues.

In this case, the user moves the biological information collecting device 100 such that the axial direction of the first acoustic waveguide 141 changes with the inserting portion 104 of the biological information collecting device 100 still held inside the acoustic foramen 200. And he or she stops moving the biological information collecting device 100 when hearing the sound most clearly. When the user presses the measuring start switch 103 once again in such a state, the microcomputer 110 activates the chopper 118, and the infrared radiation emitted from the eardrum 202 starts to be measured.

On sensing, by reference to the clock signal supplied from the timer 156, that a predetermined amount of time has passed since the measuring process was started, the microcomputer 110 controls the chopper 118 to block the infrared radiation from reaching the optical filter wheel 106. As a result, the measuring process ends automatically. At this point in time, by controlling the display 114 or the buzzer 158, the microcomputer 110 shows a message telling that the measuring process has ended on the display 114, makes the buzzer 158 beep or outputs a voice message or an alarm through a loudspeaker (not shown), thereby notifying the user of the end of the measuring process. On confirming that the measuring process has ended, the user removes the inserting portion 104 from his or her acoustic foramen 200.

Next, the microcomputer 110 reads correlation data, showing the correlation between the respective signal values of the electrical signals representing the intensities of the infrared radiations that have been transmitted through the first and second optical filters 122 and 124 and the biological constituent concentration, from the memory 112, and converts the electrical signal supplied from the A/D converter 138 into a biological constituent concentration with reference to this correlation data. The biological constituent concentration thus calculated is presented on the display 114 eventually.

According to this preferred embodiment, by moving the biological information collecting device 100 while listening to the acoustic wave that has been transmitted through the first acoustic waveguide 141, the user can see what the inserting portion 104 held in the acoustic foramen 200 now faces. And if the user stops moving the biological information collecting device 100 when he or she hears the sound most clearly, the measuring process can get done with the end of the inserting portion 104 that is held inside the acoustic foramen 200 facing the eardrum 202 (i.e., with the field of view of the infrared sensor 108 directed toward the eardrum). As a result, the biological information can be collected even more accurately.

### Embodiment 2

Hereinafter, a biological information collecting device as a second preferred embodiment of the present invention will be described.

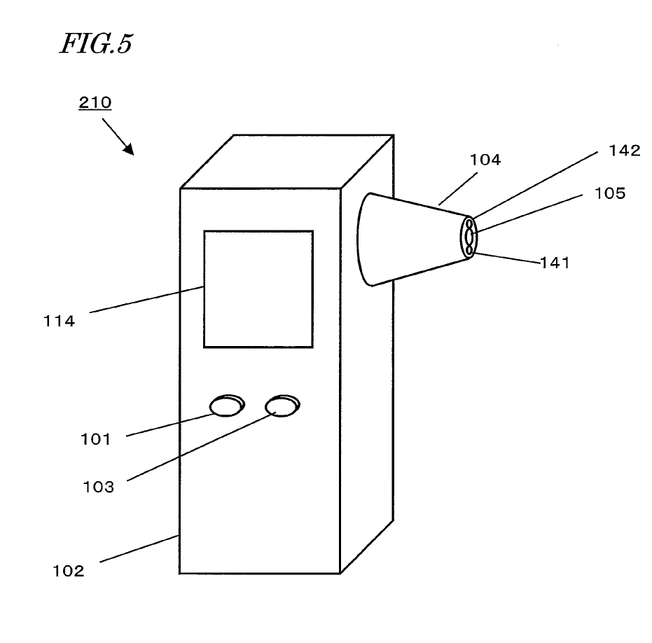

The appearance of the biological information collecting device 210 of this preferred embodiment is different from that of the first preferred embodiment described above in that the device 210 further includes a second acoustic waveguide 142. Other than that, the appearance of the second preferred embodiment is quite the same as the first preferred embodiment, and the description thereof will be omitted herein. FIG. 5 is a perspective view illustrating the appearance of the biological information collecting device 210 of the second preferred embodiment.

The inserting portion 104 includes an optical waveguide 105 for guiding the infrared radiation that has been emitted from inside the acoustic foramen into the biological information collecting device 210, a first acoustic waveguide 141 for transmitting an acoustic wave from the body 102 into the acoustic foramen, and a second acoustic waveguide 142 for guiding the wave that has been reflected back from inside the acoustic foramen toward the body.

In this example, the respective openings of the first and second acoustic waveguides 141 and 142 are located at the end (or terminal) of the inserting portion 104. Thus, if the inserting portion 104 is inserted into the acoustic foramen so that the end of the inserting portion 104 faces the eardrum, then the openings of the first and second acoustic waveguides 141 and 142 also face the eardrum. The first and second acoustic waveguides 141 and 142 respectively correspond to the first and second acoustic waveguides of the present invention. Anything (such as a hollow tube) may be used as the second acoustic waveguide as long as the waveguide can guide the acoustic wave.

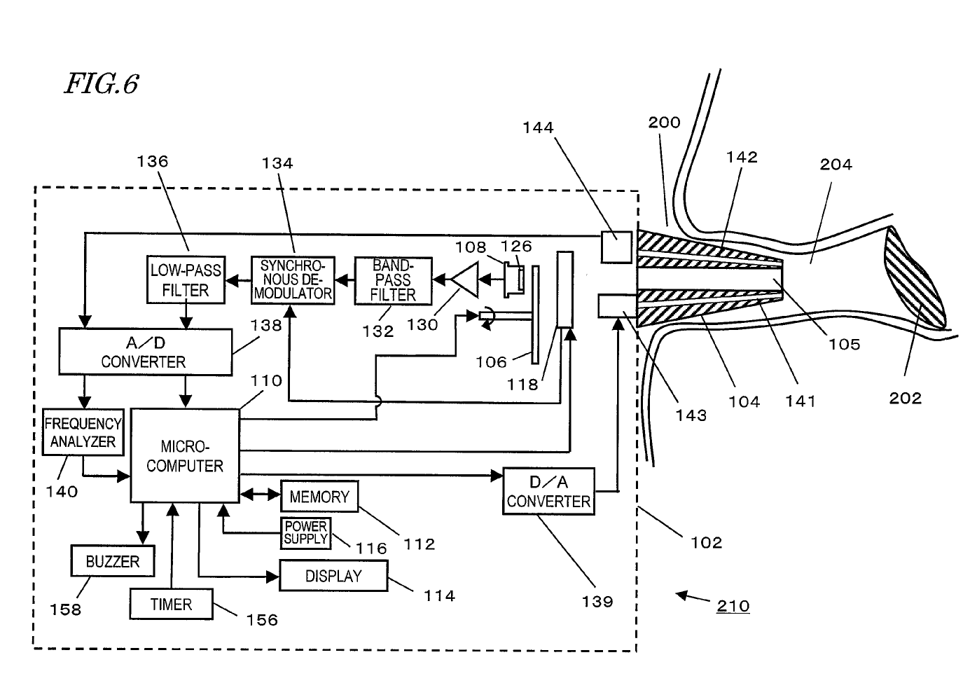

Next, the internal configuration of the body of the biological information collecting device 210 will be described with reference to FIG. 6, which illustrates the configuration of the biological information collecting device 210 of the second preferred embodiment.

The biological information collecting device 210 of this preferred embodiment further includes a second acoustic waveguide 142, a microphone 144 and a frequency analyzer 140 in addition to every component of the first preferred embodiment described above. However, the other components are quite the same as the counterparts of the first preferred embodiment, and the description thereof will be omitted herein.

The sound source 143 has the function of producing an acoustic wave to be incident onto the inside of the acoustic foramen 200. As in the first preferred embodiment described above, the sound source 143 also produces an acoustic wave in this preferred embodiment as a simple tone with a single frequency of 1,200 Hz.

The acoustic wave produced by the sound source 143 will be incident on the acoustic foramen 200 through the first acoustic waveguide 141. The acoustic wave that has been incident on the acoustic foramen 200 will be partially absorbed into, and partially reflected from, the eardrum 202, the ear canal 204 and other vital tissues. When the acoustic wave is reflected from the vital tissues, reflected waves are produced. Among those reflected waves produced inside the acoustic foramen 200, the reflected wave that has returned to the inserting portion 104 is guided back through the second acoustic waveguide 142 into the body 102.

The microphone 144 has the function of converting the reflected wave that has been guided back through the second acoustic waveguide 142 to the body 102 into an electrical signal. In this case, the microphone 144 corresponds to an acoustic wave detector according to the present invention.

As the acoustic wave detector, any known acoustic wave detector may be used without particular restriction. However, a microphone with a uniform, sharp or super directivity is particularly preferred and it preferably has a small size. Specifically, a capacitor microphone (e.g., an electret capacitor microphone, among other things) is preferred. Also, in order to avoid directly detecting the acoustic wave that has been produced from the sound source, the microphone is preferably arranged somewhere other than the area where it has sensitivity, and is preferably covered with some acoustical material entirely but its acoustic wave detecting area. As the acoustical material, a urethane foam, a nonwoven fabric or any other known material may be used without restriction.

The sound source 143 is arranged in an area where the microphone 144 has no sensitivity. A unidirectional microphone, for example, has no sensitivity in the rear of its sensor portion. That is why when a unidirectional microphone is used, the sound source may be arranged behind the sensor portion of the microphone. On the other hand, a microphone with sharp or super directivity has no sensitivity beside its sensor portion. In this preferred embodiment, a microphone 144 with sharp directivity is used and the sound source 143 is arranged beside the microphone 144.

The output electrical signal of the microphone 144 is converted by the A/D converter 138 into a digital signal, which is then supplied to the frequency analyzer 140.

The frequency analyzer 140 has the function of classifying the output electrical signals of the A/D converter 138 according to the frequency and outputting them to the microcomputer 110. As the frequency analyzer 140, an LSI (large scale integrated circuit) with fast Fourier transform function may be used. For example, a speech recognition LSI may be used. By using the frequency analyzer 140, the frequency components of the acoustic wave that has been detected by the microphone 144 can be analyzed. That is why if the microcomputer 110 identifies acoustic waves with frequency components that do not agree with those of the acoustic wave produced by the sound source 143 and removes such acoustic waves from the one detected by the microphone 144, then the influence of unnecessary frequency components can be reduced.

The reflected wave that has been guided into the body 102 is converted by the microphone 144 into an electrical signal. Then, the electrical signal converted from the reflected wave is further converted by the A/D converter into a digital signal. Then, the electrical signal that has been converted into a digital signal is subjected to an analysis by the frequency analyzer 140 to find the frequencies of the acoustic waves that have been included in the reflected wave. Since the sound source 143 produces only an acoustic wave with a frequency of 1,200 Hz, acoustic waves with other frequencies will become noise. And by getting an electrical signal representing such noise removed by a band-pass filter circuit built in the microcomputer 110, the electrical signal representing the reflected wave can be extracted inside the microcomputer 110.

In the memory 112, stored is a threshold value for the electrical signal representing the intensity of the reflected wave that has been detected by the microphone 144.

The microcomputer 110 reads the threshold value from the memory 112 and compares it to the electrical signal supplied from the frequency analyzer 140 to represent the intensity of the reflected wave.

If the end face of the inserting portion 104 is opposed to the eardrum 202, the acoustic wave that has been transmitted through the first acoustic waveguide 141 inside the acoustic foramen 200 reaches the eardrum 202. When the acoustic wave has a frequency of 1,200 Hz, the acoustic wave is reflected from the eardrum 202 at a reflectance of about 0.5 as shown in FIG. 4. As a result, the ratio of the intensity of the acoustic wave to that of the reflected wave detected by the microphone 144 decreases. In this preferred embodiment, as settings are done to keep the intensity of the acoustic wave constant, the intensity of the reflected wave detected by the microphone 144 becomes minimum when the inserting portion faces the eardrum.

On the other hand, the acoustic wave with a frequency of 1,200 Hz is reflected from the ear canal 204 at as high a reflectance as approximately 0.9 (not shown). That is why if the end face of the inserting portion 104 is opposed to the ear canal 204, the ratio of the intensity of the acoustic wave to that of the reflected wave detected by the microphone 144 increases. In this preferred embodiment, as settings are done to keep the intensity of the acoustic wave constant, the intensity of the reflected wave detected by the microphone 144 increases to exceed the one in the situation where the end face of the inserting portion 104 is opposed to the eardrum 202.

The threshold value stored in the memory 112 is set somewhere between the respective intensities of the reflected waves detected by the microphone 144 in a situation where the end face of the inserting portion 104 is opposed to the eardrum 202 and in a situation where the end face of the inserting portion 104 is opposed to the ear canal 204.

The microcomputer 110 reads the threshold value from the memory 112 and compares it to the electrical signals that have been supplied from the frequency analyzer 140 to represent the intensities of the reflected waves.

If the microcomputer 110 finds the intensity of the reflected wave detected by the microphone 144 greater than the threshold value as a result of comparison, then it can be determined that the end face of the inserting portion 104 is opposed to the ear canal 204, not the eardrum 202. As a result, it can be seen that the inserting portion 104 has been introduced into the acoustic foramen 200 improperly.

In that case, the microcomputer 110 controls the buzzer 158 to make the buzzer 158 beep. In this manner, the user can be notified that the inserting portion 104 has been introduced into the acoustic foramen 200 improperly, and can be prompted to change the directions of the inserting portion 104 inside the acoustic foramen 200. The buzzer 158 corresponds to an alarm output section according to the present invention. Alternatively, the alarm output section may also be a display that shows a text warning or a loudspeaker that outputs an alarm sound or voice message.

If the buzzer 158 has beeped, the user changes the directions of the inserting portion 104 inside the acoustic foramen 200 so that the end face of the inserting portion 104 is opposed to the eardrum 202. In that case, the user may change the positions of the inserting portion 104 so that he or she can hear the acoustic wave sound more clearly.

If the microcomputer 110 finds the intensity of the reflected wave detected by the microphone 144 smaller than the threshold value as a result of comparison, then it can be determined that the end face of the inserting portion 104 is opposed to the eardrum 202. As a result, it can be seen that the inserting portion 104 has been introduced into the acoustic foramen 200 properly. The microcomputer 110 corresponds to a decision section according to the present invention. As the decision section, a logic circuit may be used, for example.

In that case, the microcomputer 110 controls the buzzer 158 to make the buzzer 158 emit a different sound from the alarm. On determining that the end face of the inserting portion 104 is opposed to the eardrum 202, the microcomputer 110 activates the chopper 118, thereby automatically starting to measure the infrared radiation emitted from the eardrum 202. By sounding the buzzer 158, the user can be notified that the inserting portion 104 has been introduced into the acoustic foramen 200 properly and that the measuring process has started.

In this case, the ringing sound may be any sort of sound as long as the frequency, the duration or the number of times of ringing of that sound is different from that of the alarm to the point that the user can easily tell the ringing sound from the alarm. For example, the duration of the ringing sound may be shorter than that of the alarm.

On sensing, by reference to the clock signal supplied from the timer 156, that a predetermined amount of time has passed since the measuring process was started, the microcomputer 110 controls the chopper 118 to block the infrared radiation from reaching the optical filter wheel 106. As a result, the measuring process ends automatically. At this point in time, by controlling the display 114 or the buzzer 158, the microcomputer 110 shows a message telling that the measuring process has ended on the display 114, makes the buzzer 158 beep, or outputs a voice message or an alarm through a loudspeaker (not shown), thereby notifying the user of the end of the measuring process. On confirming that the measuring process has ended, the user removes the inserting portion 104 from his or her acoustic foramen 200.

Next, the microcomputer 110 reads correlation data, showing the correlation between the respective signal values of the electrical signals representing the intensities of the first and second infrared radiations that have been transmitted through the first and second optical filters 122 and 124 and the biological constituent concentration, from the memory 112, and converts the electrical signal supplied from the A/D converter 138 into a biological constituent concentration with reference to this correlation data. The biological constituent concentration thus calculated is presented on the display 114 eventually.

According to this preferred embodiment, by comparing the intensity of the reflected wave to the threshold value, the user can see what the inserting portion 104 held in the acoustic foramen 200 now faces. Since the biological information collecting device 210 automatically determines whether the field of view of the infrared sensor 108 faces the eardrum 202 or not, there is no need for the user to make that decision by him- or herself. In addition, since the measuring process can get done with the end of the inserting portion 104 that is held inside the acoustic foramen 200 facing the eardrum 202, the biological information can be collected even more accurately.

Optionally, the device may be modified so as to allow the user to make the decision by him- or herself as in the first preferred embodiment described above. However, it could be hard for some users to hear an acoustic wave with a high frequency. In that case, the acoustic wave with the high frequency may be changed into an acoustic wave with a lower frequency. Then, it can be determined more accurately whether or not the user can hear the acoustic wave more clearly.

### Embodiment 3

Hereinafter, a biological information collecting device as a third preferred embodiment of the present invention will be described.

The appearance of the biological information collecting device 211 of this preferred embodiment is the same as that of the biological information collecting device 210 of the second preferred embodiment described above, and the description thereof will be omitted herein.

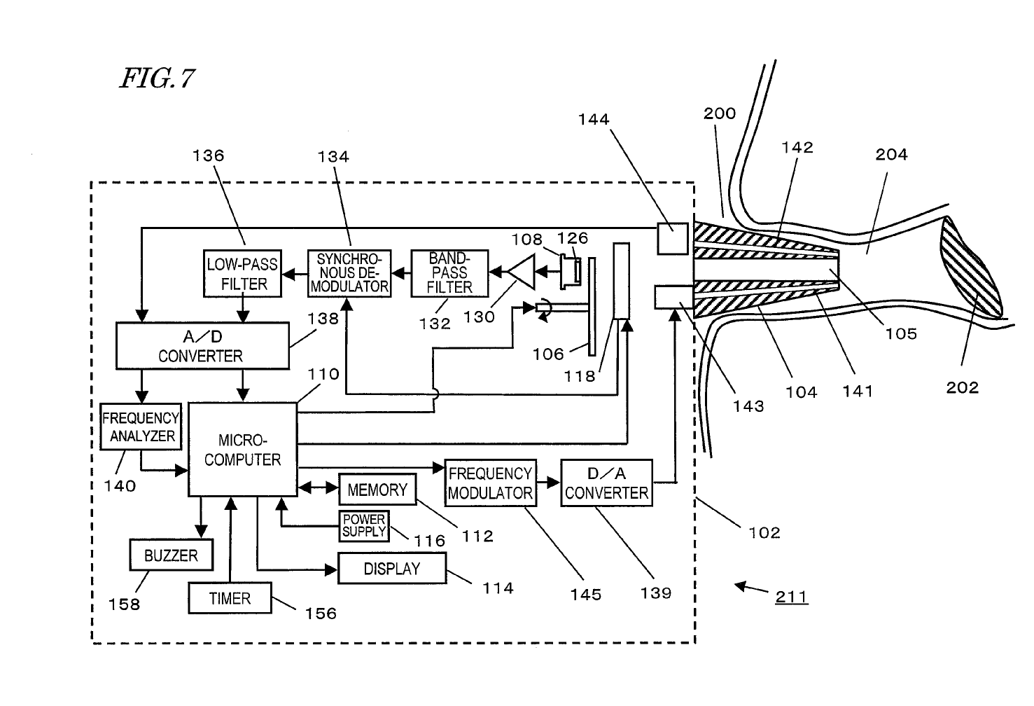

Next, the internal configuration of the body of the biological information collecting device 211 will be described with reference to FIG. 7, which illustrates the configuration of the biological information collecting device 211 of the third preferred embodiment. The biological information collecting device 211 of this preferred embodiment further includes a frequency modulator 145 in addition to every component of the second preferred embodiment described above.

The body of the biological information collecting device 211 includes a chopper 118, an optical filter wheel 106, an infrared sensor 108, a pre-amplifier 130, a band-pass filter 132, a synchronous demodulator 134, a low-pass filter 136, an analog-to-digital (A/D) converter 138, a microcomputer 110, a memory 112, a display 114, a power supply 116, a timer 156, a sound source 143, a digital-to-analog (D/A) converter 139, a frequency modulator 145, a microphone 144, a frequency analyzer 140 and a buzzer 158. The microcomputer 110 corresponds to the computing section and the control section of the present invention.

The power supply 116 supplies alternating current (AC) or direct current (DC) power to the microcomputer 110. A battery is preferably used as the power supply 116.

The sound source 143 has the function of producing an acoustic wave to beat on the acoustic foramen 200. The frequency of the acoustic wave produced by the sound source 143 is adjusted by the frequency modulator 145 to a desired one. The output digital signal of the frequency modulator 145 is converted by the D/A converter 138 into an analog signal, which is then passed to the sound source 143. Then the sound source 143 produces the acoustic wave based on the analog signal received. The operations of the sound source 143 and the frequency modulator 145 are controlled in accordance with a control signal supplied from the microcomputer 110.

In this preferred embodiment, the sound source 143 produces a first acoustic wave as a simple tone with a single frequency of 400 Hz and a second acoustic wave as a simple tone with a single frequency of 1,200 Hz.

The first and second acoustic waves produced by the sound source 143 will be incident on the acoustic foramen 200 through the first acoustic waveguide 141. The first and second acoustic waves that have been incident on the acoustic foramen 200 will be partially absorbed into, and partially reflected from, the eardrum 202, the ear canal 204 and other vital tissues. When the first and second acoustic waves are reflected from the vital tissues, first and second reflected waves are produced, respectively. Among the first and second reflected waves produced inside the acoustic foramen 200, the reflected wave that has returned to the inserting portion 104 is guided back through the second acoustic waveguide 142 into the body 102.

The microphone 144 has the function of converting the first and second reflected waves that have been guided back through the second acoustic waveguide 142 to the body 102 into electrical signals. In this case, the microphone 144 corresponds to an acoustic wave detector according to the present invention.

The sound source 143 is arranged in an area where the microphone 144 has no sensitivity. A unidirectional microphone, for example, has no sensitivity in the rear of its sensor portion. That is why when a unidirectional microphone is used, the sound source may be arranged behind the sensor portion of the microphone. On the other hand, a microphone with sharp or super directivity has no sensitivity beside its sensor portion. In this preferred embodiment, a microphone 144 with sharp directivity is used and the sound source 143 is arranged beside the microphone 144.

The output electrical signal of the microphone 144 is converted by the A/D converter 138 into a digital signal, which is then supplied to the frequency analyzer 140.

The frequency analyzer 140 has the function of classifying the output electrical signals of the A/D converter 138 according to the frequency and outputting them to the microcomputer 110. As the frequency analyzer 140, an LSI (large scale integrated circuit) with fast Fourier transform function may be used. For example, a speech recognition LSI may be used. By using the frequency analyzer 140, the frequency components of the acoustic wave that has been detected by the microphone 144 can be analyzed. That is why if the microcomputer 110 identifies acoustic waves with frequency components that do not agree with those of the first and second acoustic waves and removes such acoustic waves from the one detected by the microphone 144, then the influence of unnecessary frequency components can be reduced.

In the memory 112, stored are a first threshold value for the electrical signal representing the intensity of the first reflected wave that has been detected by the microphone 144 and a second threshold value for the electrical signal representing the intensity of the second reflected wave that has been detected by the microphone 144. The memory 112 corresponds to a threshold value storage section according to the present invention. As the threshold value storage section, a memory such as a RAM or a ROM may be used, for example.

The microcomputer 110 reads the first and second threshold values from the memory 112 and compares them to the respective electrical signals supplied from the frequency analyzer 140 to represent the intensities of the first and second reflected waves. The microcomputer 110 corresponds to a comparison section according to the present invention. As the comparison section, a logic circuit may be used, for example.

The chopper 118 chops the infrared radiation that has been emitted from the eardrum 202 and then guided into the body 102 through the optical waveguide 105, thereby transforming the infrared radiation into a high-frequency infrared signal. The operation of the chopper 118 is controlled in accordance with a control signal supplied from the microcomputer 110. The infrared radiation that has been chopped by the chopper 118 soon reaches the optical filter wheel 106.

In the optical filter wheel 106, first and second optical filters 122 and 124 are fitted into a ring 123 as shown in FIG. 3. In the example illustrated in FIG. 3, the first and second optical filters 122 and 124, both of which are semicircular, are fitted into the ring 123, thereby forming a disklike member. And at the center of that disklike member, arranged is a shaft 125.

By rotating the shaft 125 in the direction indicated by the arrow shown in FIG. 3, the optical filters to pass the infrared radiation that has been chopped by the chopper 118 may be switched from one of these two optical filters 122 and 124 into the other. The rotation of the shaft 125 is controlled in accordance with a control signal supplied from the microcomputer 110.

The shaft 125 preferably has its revolution synchronized with the rotation of the chopper 118 and is preferably controlled so as to turn 180 degrees while the chopper 118 is closed. This is because in that case, when the chopper 118 is opened next time, the infrared radiation to be chopped by the chopper 118 may be transmitted through the next optical filter. The optical filter wheel 106 corresponds to a spectral element according to the present invention.

The infrared radiation that has been transmitted through the first or second optical filter 122 or 124 reaches the infrared sensor 108 with a sensing area 126. On reaching the infrared sensor 108, the infrared radiation is incident on the sensing area 126. The infrared sensor 108 receives the infrared radiation and transforms the infrared radiation into an electrical signal representing its intensity.

The electrical signal is output from the infrared sensor 108 to the pre-amplifier 130 and then amplified there. Then, the amplified electrical signal has its signal components filtered out by the band-pass filter 132 except those falling within a frequency range, of which the center frequency is defined by the chopping frequency. As a result, noise caused by some statistical fluctuation such as thermal noise can be minimized.

The electrical signal that has been subjected to the filtering process by the band-pass filter 132 is synchronized with the chopping frequency of the chopper 118 and integrated by the synchronous demodulator 134 so as to be demodulated into a DC signal.

Next, the electrical signal that has been demodulated by the synchronous demodulator 134 has its high frequency components filtered out by the low-pass filter 136. In this manner, its noise can be further reduced.

Subsequently, the electrical signal that has been subjected to the filtering process by the low-pass filter 136 is converted by the A/D converter 138 into a digital signal, which is then input to the microcomputer 110. In this case, the electrical signal that has come from any of the optical filters by way of the infrared sensor 108 can have its source identified (i.e., it is possible to determine which of those optical filters the infrared radiation, represented by the electrical signal, has been transmitted through) by using a control signal for the shaft 125 as a trigger. The duration of an electrical signal associated with the same optical filter is defined as an interval after the microcomputer 110 has output a control signal for the shaft 125 and before it outputs the next shaft control signal. By calculating the integral of the electrical signals associated with the respective optical filters on the memory 112 and then working out its average, the noise can be further reduced. That is why the measured values are preferably integrated.

In the memory 112, stored is correlation data that shows a correlation between the signal values of the electrical signals representing the respective intensities of the infrared radiations transmitted through the first and second optical filters 122 and 124 and the biological constituent concentration. The microcomputer 110 reads this concentration correlation data from the memory 112, calculates a digital signal per unit time based on the digital signal that has been stored in the memory 112 by reference to the correlation data, and converts the digital signal into a biological constituent concentration. The memory 112 corresponds to a correlation data storage section according to the present invention.

Then, the biological constituent concentration that has been worked out by the microcomputer 110 is output to and presented on the display 114. The display 114 corresponds to a display section according to the present invention.

The first optical filter 122 has such a spectral characteristic as to transmit infrared radiation that falls within a wavelength range including the wavelength to be absorbed into the biological constituent under measurement (which will be referred to herein as “measuring wavelength range”). On the other hand, the second optical filter 124 has a different spectral characteristic from the first optical filter's 122. Specifically, the second optical filter 124 has such a spectral characteristic as to transmit infrared radiation that falls within a wavelength range including a wavelength to be absorbed into not the biological constituent under measurement but another biological constituent that would interfere with the measurement of the target biological constituent (which will be referred to herein as “reference wavelength range”). In this case, that another biological constituent may be any constituent that is included a lot in the organism other than the biological constituent under measurement.

For example, glucose has an infrared absorption spectrum with a peak of absorption around 9.6 μm. That is why if the biological constituent under measurement is glucose, the first optical filter 122 preferably has such a spectral characteristic as to transmit infrared radiation that falls within a wavelength range including 9.6 μm.

Meanwhile, protein, included a lot in an organism, would absorb infrared radiation around 8.5 μm, while glucose would not absorb infrared radiation around that wavelength. That is why the second optical filter 124 preferably has such a spectral characteristic as to transmit infrared radiation that falls within a wavelength range including 8.5 μm.

The correlation data stored in the memory 112 to show the correlation between the respective signal values of the electrical signals representing the intensities of the infrared radiations that have been transmitted through the first and second optical filters 122 and 124 and the biological constituent concentration may be obtained in the following manner, for example.

First, as for a patient with a known biological constituent concentration such as a blood glucose level, the infrared radiation that has been emitted from his or her eardrum 202 has its intensity measured. In this case, electrical signals representing the intensities of infrared radiations falling within the wavelength ranges to be transmitted by the first and second optical filters 122 and 124 are obtained. By making such measurement on a number of patients with mutually different biological constituent concentrations, multiple sets of data, each including the electrical signals representing the intensities of infrared radiations falling within the wavelength ranges to be transmitted by the first and second optical filters 122 and 124 and their associated biological constituent concentrations, can be collected.

Next, by analyzing these data sets that have been collected in this manner, correlation data is obtained. For example, a multivariate analysis is carried out by either a multiple regression analysis such as partial least squares regression (PLS) method or a neural network method on the electrical signals representing the intensities of infrared radiations falling within the wavelength ranges to be transmitted by the first and second optical filters 122 and 124 and their associated biological constituent concentrations. As a result, a function showing a correlation between the electrical signals representing the intensities of infrared radiations falling within the wavelength ranges to be transmitted by the first and second optical filters 122 and 124 and their associated biological constituent concentrations can be obtained.

Also, the first optical filter 122 may have such a spectral characteristic as to transmit infrared radiation falling within a measuring wavelength range and the second optical filter 124 may have such a spectral characteristic as to transmit infrared radiation falling within a reference wavelength range. In that case, the difference between the signal values of the electrical signals representing the intensities of infrared radiations falling within the wavelength ranges to be transmitted by the first and second optical filters 122 and 142 may be calculated, and the correlation between that difference and its associated biological constituent concentration may be obtained as the correlation data by performing a linear regression analysis such as a minimum square method, for example.

Hereinafter, it will be described with reference to FIGS. 3, 5 and 7 how the biological information collecting device 211 of this preferred embodiment operates.

First, when the user presses the power switch 101 of the biological information collecting device 211, the power is turned ON inside the body 102 to get the biological information collecting device 211 ready to make measurements.

Next, the user holds the body 102 in his or her hand to introduce the inserting portion 104 into his or her ear canal 204 as shown in FIG. 7. In this case, the inserting portion 104 is introduced such that the tip of the optical waveguide 105 is directed toward the eardrum 202. The inserting portion 104 is a conical hollow tube that increases its diameter from the tip of the inserting portion toward the portion connected to the body 102. That is why the inserting portion 104 has such a structure as to prevent itself from being inserted any deeper than the position where the outside diameter of the inserting portion gets equal to the inside diameter of the acoustic foramen 200.

Subsequently, when the user presses the measuring start switch 103 of the biological information collecting device 211 with the biological information collecting device 211 held at the position where the outside diameter of the inserting portion 104 gets equal to the inside diameter of the acoustic foramen 200, the microcomputer 110 activates the frequency modulator 145, thereby producing first and second acoustic waves from the sound source 143. In this example, the sound source 143 produces a first acoustic wave as a simple tone with a single frequency of 400 Hz and a second acoustic wave as a simple tone with a single frequency of 1,200 Hz at a constant intensity for one second each. Settings have been done so that the intensities of the first and second acoustic waves are equal to each other.

The acoustic waves that have been produced by the sound source 143 are transmitted through the first acoustic waveguide 141 and into the acoustic foramen 200. Then, the first and second acoustic waves propagating through the acoustic foramen 200 are partially absorbed into, and partially reflected from, the eardrum 202, the ear canal 204 and other vital tissues. The first and second acoustic waves are reflected from the vital tissues to produce first and second reflected waves, respectively. Parts of the first and second reflected waves that have been produced inside the acoustic foramen 200 return to the inserting portion 104 and then are guided through the second acoustic waveguide 142 into the body 102.

The first and second reflected waves that have been guided into the body 102 are converted by the microphone 144 into electrical signals. Then, the electrical signals converted from the reflected waves are further converted by the A/D converter into digital signals. Thereafter, the electrical signals that have been converted into digital signals are subjected to an analysis by the frequency analyzer 140 to find the frequencies of the acoustic waves that have been included in the reflected waves. Since the sound source 143 produces only acoustic waves with frequencies of 400 Hz and 1,200 Hz, acoustic waves with other frequencies will become noise. And by getting an electrical signal representing such noise removed by a band-pass filter circuit built in the microcomputer 110, the electrical signals representing the first and second reflected waves can be extracted inside the microcomputer 110.

In the memory 112, stored are first and second threshold values for the electrical signals representing the respective intensities of the first and second reflected waves that have been detected by the microphone 144.

The microcomputer 110 reads the first and second threshold values from the memory 112 and compares them to the electrical signals supplied from the frequency analyzer 140 to represent the respective intensities of the first and second reflected waves.

If the end face of the inserting portion 104 is opposed to the eardrum 202, the first and second acoustic waves that have been transmitted through the first acoustic waveguide 141 inside the acoustic foramen 200 reach the eardrum 202. The first acoustic wave with a frequency of 400 Hz is reflected from the eardrum 202 at a reflectance of about 0.9 as shown in FIG. 4. On the other hand, the second acoustic wave with a frequency of 1,200 Hz is reflected from the eardrum 202 at a reflectance of about 0.5 as shown in FIG. 4. As a result, the ratio of the intensity of the first acoustic wave to that of the first reflected wave detected by the microphone 144 becomes greater than the ratio of the intensity of the second acoustic wave to that of the second reflected wave detected by the microphone 144. In this preferred embodiment, settings are done so that the intensities of the first and second acoustic waves are equal to each other. Consequently, the intensity of the first reflected wave detected by the microphone 144 becomes greater than that of the second reflected wave detected by the microphone 144.

As for the reflectances of the acoustic waves from the ear canal 204, on the other hand, the first and second acoustic waves with frequencies of 400 Hz and 1,200 Hz both have reflectances as high as about 0.9. That is why if the end face of the inserting portion 104 is opposed to the ear canal 204, the ratio of the intensity of the first acoustic wave to that of the first reflected wave detected by the microphone 144 and the ratio of the intensity of the second acoustic wave to that of the second reflected wave detected by the microphone 144 both increase. In this preferred embodiment, as settings are done so that the intensities of the first and second acoustic waves are equal to each other, the intensities of the first and second reflected waves detected by the microphone 144 are both high. Consequently, the intensity of the second reflected wave becomes approximately equal to that of the first reflected wave detected by the microphone 144 in a situation where the end face of the inserting portion 104 is opposed to the eardrum 202.

However, if the end face of the inserting portion 104 is opposed to neither the eardrum 202 nor the ear canal 204, then parts of the first and second reflected waves that reach the inserting portion 104 decrease. As a result, the ratio of the intensity of the first acoustic wave to that of the first reflected wave detected by the microphone 144 and the ratio of the intensity of the second acoustic wave to that of the second reflected wave detected by the microphone 144 both decrease. In this preferred embodiment, as settings are done so that the intensities of the first and second acoustic waves are equal to each other, the intensities of the first and second reflected waves detected by the microphone 144 are both low.

The first threshold value stored in the memory 112 is set somewhere between the respective intensities of the first reflected waves detected by the microphone 144 in a situation where the end face of the inserting portion 104 is opposed to the eardrum 202 and in a situation where the end face of the inserting portion 104 is opposed to neither the eardrum 202 nor the ear canal 204. On the other hand, the second threshold value stored in the memory 112 is set somewhere between the respective intensities of the second reflected waves detected by the microphone 144 in a situation where the end face of the inserting portion 104 is opposed to the ear canal 204 and in a situation where the end face of the inserting portion 104 is opposed to the eardrum 202.

The microcomputer 110 reads the first and second threshold values from the memory 112 and compares them to the electrical signals that have been supplied from the frequency analyzer 140 to represent the respective intensities of the first and second reflected waves.

If the microcomputer 110 finds the intensity of the first reflected wave detected by the microphone 144 equal to or smaller than the first threshold value or the intensity of the second reflected wave detected by the microphone 144 equal to or greater than the second threshold value as a result of comparison, then it can be determined that the end face of the inserting portion 104 is opposed to the ear canal 204, not the eardrum 202, or neither the eardrum 202 nor the ear canal 204. As a result, it can be seen that the inserting portion 104 has been introduced into the acoustic foramen 200 improperly.

In that case, the microcomputer 110 controls the buzzer 158 to make the buzzer 158 beep. In this manner, the user can be notified that the inserting portion 104 has been introduced into the acoustic foramen 200 improperly, and can be prompted to change the directions of the inserting portion 104 inside the acoustic foramen 200. The buzzer 158 corresponds to an alarm output section according to the present invention.

If the buzzer 158 has beeped, the user changes the directions of the inserting portion 104 inside the acoustic foramen 200 so that the end face of the inserting portion 104 is opposed to the eardrum 202. In that case, the user may change the positions of the inserting portion 104 so that he or she can hear a sound corresponding to the second acoustic wave, having a higher interval than the first acoustic wave, more clearly.

If the microcomputer 110 finds the intensity of the first reflected wave detected by the microphone 144 greater than the first threshold value and the intensity of the second reflected wave detected by the microphone 144 smaller than the second threshold value as a result of comparison, then it can be determined that the end face of the inserting portion 104 is opposed to the eardrum 202. As a result, it can be seen that the inserting portion 104 has been introduced into the acoustic foramen 200 properly. The microcomputer 110 corresponds to a decision section according to the present invention.

In that case, the microcomputer 110 controls the buzzer 158 to make the buzzer 158 emit a different sound from the alarm. On determining that the end face of the inserting portion 104 is opposed to the eardrum 202, the microcomputer 110 activates the chopper 118, thereby automatically starting to measure the infrared radiation emitted from the eardrum 202. By sounding the buzzer 158, the user can be notified that the inserting portion 104 has been introduced into the acoustic foramen 200 properly and that the measuring process has started.

In this case, the ringing sound may be any sort of sound as long as the frequency, the duration or the number of times of ringing of that sound is different from that of the alarm to the point that the user can easily tell the ringing sound from the alarm. For example, the duration of the ringing sound may be shorter than that of the alarm.

On sensing, by reference to the clock signal supplied from the timer 156, that a predetermined amount of time has passed since the measuring process was started, the microcomputer 110 controls the chopper 118 to block the infrared radiation from reaching the optical filter wheel 106. As a result, the measuring process ends automatically. At this point in time, by controlling the display 114 or the buzzer 158, the microcomputer 110 shows a message telling that the measuring process has ended on the display 114, makes the buzzer 158 beep, or outputs a voice message or an alarm through a loudspeaker (not shown), thereby notifying the user of the end of the measuring process. On confirming that the measuring process has ended, the user removes the inserting portion 104 from his or her acoustic foramen 200.

Next, the microcomputer 110 reads correlation data, showing the correlation between the respective signal values of the electrical signals representing the intensities of the first and second infrared radiations that have been transmitted through the first and second optical filters 122 and 124 and the biological constituent concentration, from the memory 112, and converts the electrical signal supplied from the A/D converter 138 into a biological constituent concentration with reference to this correlation data. The biological constituent concentration thus calculated is presented on the display 114 eventually.

According to this preferred embodiment, by comparing the intensities of the first and second reflected waves to the first and second threshold values, respectively, the user can see more exactly what the inserting portion 104 held in the acoustic foramen 200 now faces. In addition, since the measurements can be made with the end face of the inserting portion 104, introduced into the acoustic foramen 200, opposed right to the eardrum 202, the biological information can be collected even more accurately.

### Embodiment 4

Hereinafter, a biological information collecting device as a fourth preferred embodiment of the present invention will be described.

The configuration of the biological information collecting device of this preferred embodiment is different from that of the biological information collecting device 211 of the third preferred embodiment described above only in the threshold values stored in the memory 112.

Specifically, in the memory 112 of the biological information collecting device 211 of the third preferred embodiment, stored are a first threshold value for an electrical signal representing the intensity of the first reflected wave that has been detected by the microphone 144 and a second threshold value for an electrical signal representing the intensity of the second reflected wave that has also been detected by the microphone 144. Instead, the memory 112 of the biological information collecting device of this preferred embodiment stores a threshold value for an electrical signal representing the difference between the respective intensities of the first and second reflected waves that have been detected by the microphone 144. In the other respects, the biological information collecting device of this preferred embodiment is quite the same as the counterpart 211 of the third preferred embodiment, and the description thereof will be omitted herein.

Hereinafter, it will be described how the biological information collecting device of this preferred embodiment operates. In the following description, the biological information collecting device 211 shown in FIG. 7 is supposed to be used.

First, when the user presses the power switch 101 (see FIG. 5) of the biological information collecting device 211, the power is turned ON inside the body 102 to get the biological information collecting device 211 ready to make measurements as in the third preferred embodiment described above.

Next, the user holds the body 102 in his or her hand to introduce the inserting portion 104 into his or her ear canal 204.

Subsequently, when the user presses the measuring start switch 103 of the biological information collecting device 211 with the biological information collecting device 211 held at the position where the outside diameter of the inserting portion 104 gets equal to the inside diameter of the acoustic foramen 200, the microcomputer 110 activates the frequency modulator 145 as in the third preferred embodiment described above, thereby producing first and second acoustic waves from the sound source 143. The frequencies, intervals, intensities and so on of the first and second acoustic waves are the same as the ones already described for the third preferred embodiment.

As in the third preferred embodiment, the acoustic waves that have been produced by the sound source 143 are transmitted through the first acoustic waveguide 141 and into the acoustic foramen 200. Parts of the acoustic waves are reflected from the vital tissues inside the acoustic foramen 200. Parts of the first and second reflected waves that have been produced inside the acoustic foramen 200 return to the inserting portion 104, are guided through the second acoustic waveguide 142 into the body 102 and then are converted by the microphone 144 into electrical signals. Then, the electrical signals converted from the reflected waves are further converted by the A/D converter 138 into digital signals. Thereafter, the digital signals are subjected to an analysis by the frequency analyzer 140 to find the frequencies of the acoustic waves that have been included in the reflected waves. By getting a digital signal representing noise (i.e., acoustic waves with frequencies other than 400 Hz and 1,200 Hz) removed by a band-pass filter circuit built in the microcomputer 110, the electrical signals representing the first and second reflected waves can be extracted inside the microcomputer 110.

In the memory 112, stored is a threshold value for an electrical signal representing the difference between the respective intensities of the first and second reflected waves that have been detected by the microphone 144.

As in the third preferred embodiment described above, if the end face of the inserting portion 104 is opposed to the eardrum 202, the intensity of the first reflected wave detected by the microphone 144 becomes greater than that of the second reflected wave detected by the microphone 144. Consequently, the difference between the electrical signals that have been output from the frequency analyzer 140 to represent the respective intensities of the first and second reflected waves comes to have a large value.

On the other hand, if the end face of the inserting portion 104 is opposed to the ear canal 204, the intensities of the first and second reflected waves detected by the microphone 144 are both high as in the third preferred embodiment described above. Consequently, the difference between the electrical signals that have been output from the frequency analyzer 140 to represent the respective intensities of the first and second reflected waves comes to have a small value.

Furthermore, if the end face of the inserting portion 104 is opposed to neither the eardrum 202 nor the ear canal 204, then the intensities of the first and second reflected waves detected by the microphone 144 are both low as in the third preferred embodiment described above. Consequently, the difference between the electrical signals that have been output from the frequency analyzer 140 to represent the respective intensities of the first and second reflected waves comes to have a small value.

The threshold value stored in the memory 112 is set somewhere between the difference between the electrical signals supplied from the frequency analyzer 140 to represent the respective intensities of the first and second reflected waves in a situation where the end face of the inserting portion 104 is opposed to the eardrum 202 and the difference between the electrical signals supplied from the frequency analyzer 140 to represent the respective intensities of the first and second reflected waves in a situation where the end face of the inserting portion 104 is not opposed to the eardrum 202.

The microcomputer 110 reads the threshold value from the memory 112 and compares it to the difference between the electrical signals that have been supplied from the frequency analyzer 140 to represent the respective intensities of the first and second reflected waves.

If the microcomputer 110 finds the difference between the electrical signals supplied from the frequency analyzer 140 to represent the respective intensities of the first and second reflected waves equal to or smaller than the threshold value as a result of comparison, then it can be determined that the end face of the inserting portion 104 is opposed to the ear canal 204, not the eardrum 202, or neither the eardrum 202 nor the ear canal 204. As a result, it can be seen that the inserting portion 104 has been introduced into the acoustic foramen 200 improperly.

In that case, the microcomputer 110 controls the buzzer 158 to make the buzzer 158 beep as in the third preferred embodiment described above. In this manner, the user can be notified that the inserting portion 104 has been introduced into the acoustic foramen 200 improperly, and can be prompted to change the directions of the inserting portion 104 inside the acoustic foramen 200.

If the buzzer 158 has beeped, the user changes the directions of the inserting portion 104 inside the acoustic foramen 200 so that the end face of the inserting portion 104 is opposed to the eardrum 202. In that case, the user may change the positions of the inserting portion 104 so that he or she can hear a sound corresponding to the second acoustic wave, having a higher interval than the first acoustic wave, more clearly as in the third preferred embodiment described above.

If the microcomputer 110 finds the difference between the electrical signals supplied from the frequency analyzer 140 to represent the respective intensities of the first and second reflected waves greater than the threshold value as a result of comparison, then it can be determined that the end face of the inserting portion 104 is opposed to the eardrum 202. As a result, it can be seen that the inserting portion 104 has been introduced into the acoustic foramen 200 properly.

In that case, the microcomputer 110 controls the buzzer 158 to make the buzzer 158 emit a different sound from the alarm as in the third preferred embodiment described above. On determining that the end face of the inserting portion 104 is opposed to the eardrum 202, the microcomputer 110 activates the chopper 118, thereby automatically starting to measure the infrared radiation emitted from the eardrum 202. By sounding the buzzer 158, the user can be notified that the inserting portion 104 has been introduced into the acoustic foramen 200 properly and that the measuring process has started.

The rest of the procedure is quite the same as the third preferred embodiment and the description thereof will be omitted herein.

According to this preferred embodiment, by comparing the difference between the respective intensities of the first and second reflected waves to the threshold value, it can be seen what the inserting portion 104 introduced into the acoustic foramen 200 now faces. In addition, since the measurements can be made with the end face of the inserting portion 104 in the acoustic foramen 200 opposed to the eardrum 202, biological information can be collected as accurately as in the third preferred embodiment described above.

### Embodiment 5

Hereinafter, a biological information collecting device as a fifth preferred embodiment of the present invention will be described.

The configuration of the biological information collecting device of this preferred embodiment is the same as that of the biological information collecting device 211 of the third preferred embodiment described above and the description thereof will be omitted herein. In the following description, the biological information collecting device 211 shown in FIG. 7 will be referred to when necessary.

First, when the user presses the power switch 101 (see FIG. 5) of the biological information collecting device 211, the power is turned ON inside the body 102 to get the biological information collecting device 211 ready to make measurements as in the third preferred embodiment described above.

Next, the user holds the body 102 in his or her hand to introduce the inserting portion 104 into his or her ear canal 204.

Subsequently, when the user presses the measuring start switch 103 of the biological information collecting device 211 with the biological information collecting device 211 held at the position where the outside diameter of the inserting portion 104 gets equal to the inside diameter of the acoustic foramen 200, the microcomputer 110 activates the frequency modulator 145 as in the third preferred embodiment described above, thereby producing first and second acoustic waves from the sound source 143. The frequencies, intervals, intensities and so on of the first and second acoustic waves are the same as the ones already described for the third preferred embodiment.

As in the third preferred embodiment, the acoustic waves that have been produced by the sound source 143 are transmitted through the first acoustic waveguide 141 and into the acoustic foramen 200. Parts of the acoustic waves are reflected from the vital tissues inside the acoustic foramen 200. Parts of the first and second reflected waves that have been produced inside the acoustic foramen 200 return to the inserting portion 104, are guided through the second acoustic waveguide 142 into the body 102 and then are converted by the microphone 144 into electrical signals. Then, the electrical signals converted from the reflected waves are further converted by the A/D converter 138 into digital signals. Thereafter, the digital signals are subjected to an analysis by the frequency analyzer 140 to find the frequencies of the acoustic waves that have been included in the reflected waves. By getting a digital signal representing noise (i.e., acoustic waves with frequencies other than 400 Hz and 1,200 Hz) removed by a band-pass filter circuit built in the microcomputer 110, the electrical signals representing the first and second reflected waves can be extracted inside the microcomputer 110.

In the memory 112, stored are first and second threshold values for the electrical signals representing the respective intensities of the first and second reflected waves that have been detected by the microphone 144.

The microcomputer 110 reads the first and second threshold values from the memory 112 and compares them to the electrical signals supplied from the frequency analyzer 140 to represent the respective intensities of the first and second reflected waves.

Unlike the third preferred embodiment described above, in the biological information collecting device 211 of this preferred embodiment, when the user presses the measuring start switch 103 of the biological information collecting device 211, not just the first and second acoustic waves are produced from the sound source 143 but also the microcomputer 110 activates the chopper 118 as well, thereby starting to measure the infrared radiation emitted from the eardrum 202, too.

On determining, based on a result of comparison between the first and second threshold values and the electrical signals supplied from the frequency analyzer 140 to represent the respective intensities of the first and second reflected waves and by reference to the clock signal supplied from the timer 156, that the sum of the periods of time in which the end face of the inserting portion 104 has been regarded, by the same criteria as the ones used in the first preferred embodiment, as being opposed to the eardrum 202 has reached a predetermined amount of time since the start of measurement, the microcomputer 110 controls the chopper 118, thereby blocking the infrared radiation from reaching the optical filter wheel 106. As a result, the measuring process ends automatically. At this point in time, by controlling the display 114 or the buzzer 158, the microcomputer 110 shows a message telling that the measuring process has ended on the display 114, makes the buzzer 158 beep or outputs a voice message or an alarm through a loudspeaker (not shown), thereby notifying the user of the end of the measuring process. On confirming that the measuring process has ended, the user removes the inserting portion 104 from his or her acoustic foramen 200.

Unlike the third preferred embodiment described above, in the biological information collecting device 211 of this preferred embodiment, the electrical signals supplied from the A/D converter 138 to represent the intensities of the first and second infrared radiations that have been transmitted through the first and second optical filters 122 and 124 are stored in the memory 112 in association with a result of comparison between the first and second threshold values and the electrical signals supplied from the frequency analyzer 140 to represent the respective intensities of the first and second reflected waves. The memory 112 corresponds to an output signal storage section according to the present invention.

The microcomputer 110 extracts, from the memory 112, only the electrical signal that was output when it was determined, by the same criteria as the ones adopted in the third preferred embodiment, that the end face of the inserting portion 104 was opposed to the eardrum 202 among the electrical signals that have been supplied from the A/D converter 138 and then stored in the memory 112. Furthermore, the microcomputer 110 reads the correlation data, showing the correlation between the electrical signals representing the respective intensities of the first and second infrared radiations that have been transmitted through the first and second optical filters 122 and 124 and the biological constituent concentration, from the memory 112 and converts the electrical signal extracted into a biological constituent concentration by reference to the correlation data. The biological constituent concentration thus obtained is presented on the display 114 then.

According to this preferred embodiment, by comparing the intensities of the first and second reflected waves to the first and second threshold values, respectively, as in the third preferred embodiment described above, it can be seen exactly what the inserting portion 104 introduced into the acoustic foramen 200 now faces. In addition, since the biological constituent concentration can be measured based on only the intensity of the infrared radiation that was detected when the end face of the inserting portion 104 in the acoustic foramen 200 was opposed to the eardrum 202, the biological information can be collected even more accurately.

### Embodiment 6

Hereinafter, a biological information collecting system will be described as a sixth preferred embodiment of the present invention.

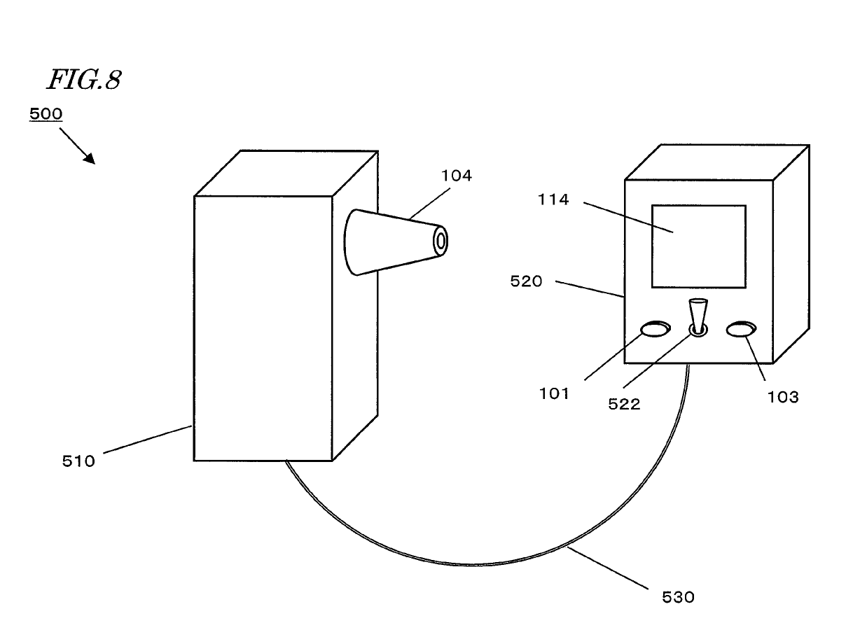

FIG. 8 is a perspective view illustrating the appearance of a biological information collecting system 500 as the sixth preferred embodiment of the present invention.

As shown in FIG. 8, the biological information collecting system 500 of this preferred embodiment includes a measuring section 510 with the inserting portion 104, and a body section 520 including the display 114, the power switch 101, the measuring start switch 103 and a direction adjustment lever switch 522. In this biological information collecting system 500, the measuring section 510 and the body section 520 are connected together with a cable 530 that transmits electrical signals.

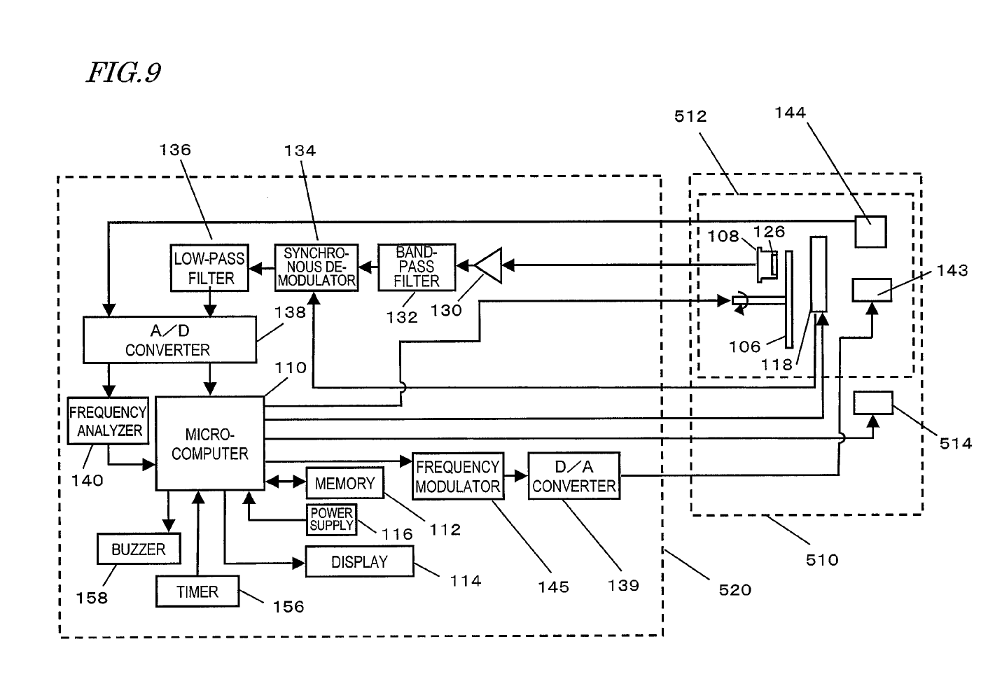

Next, the respective internal configurations of the measuring section 510 and the body section 520 of the biological information collecting system 500 will be described with reference to FIG. 9, which illustrates those configurations.

The measuring section 510 of the biological information collecting system 500 includes not only a sensing block 512 including a sound source 143, a microphone 144, a chopper 118, an optical filter wheel 106 and an infrared sensor 108 but also an adjustment section 514 for adjusting the directions of the sound source 143, the microphone 144 and the infrared sensor 108.

On the other hand, the body section 520 of the biological information collecting system 500 includes a pre-amplifier 130, a band-pass filter 132, a synchronous demodulator 134, a low-pass filter 136, an A/D converter 138, a microcomputer 110, a memory 112, a display 114, a power supply 116, a timer 156, a D/A converter 139, a frequency modulator 145, a frequency analyzer 140 and a buzzer 158.

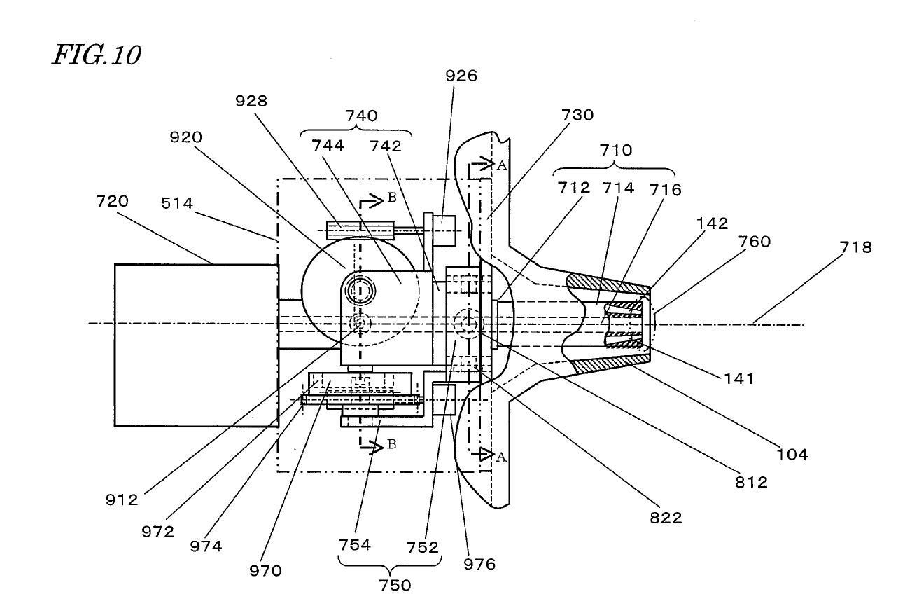

Next, the internal configuration of the measuring section 510 in the biological information collecting system 500 will be described with reference to FIGS. 10 through 13.

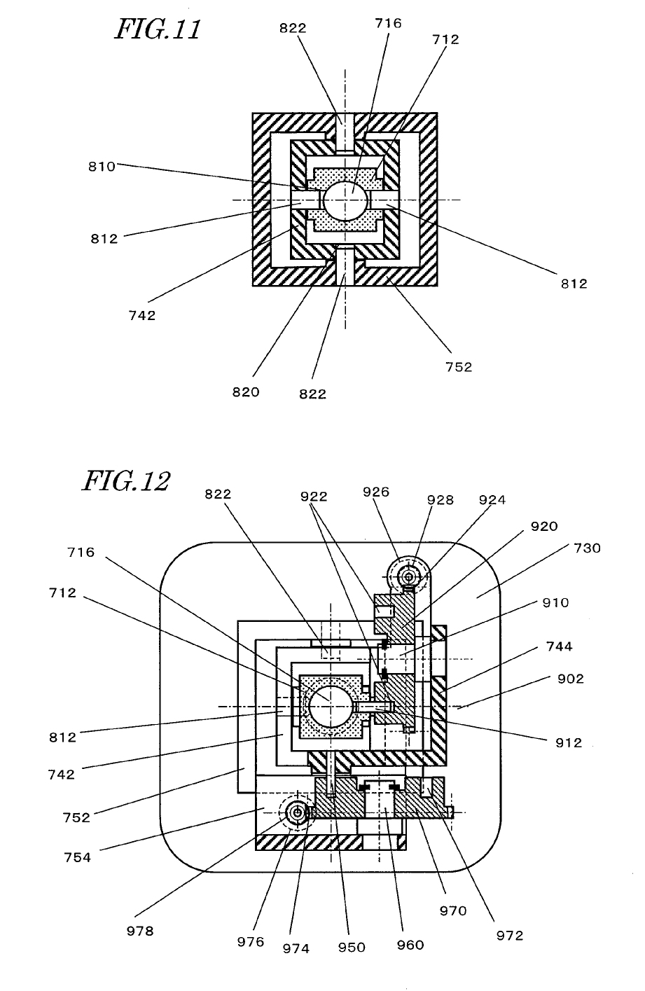

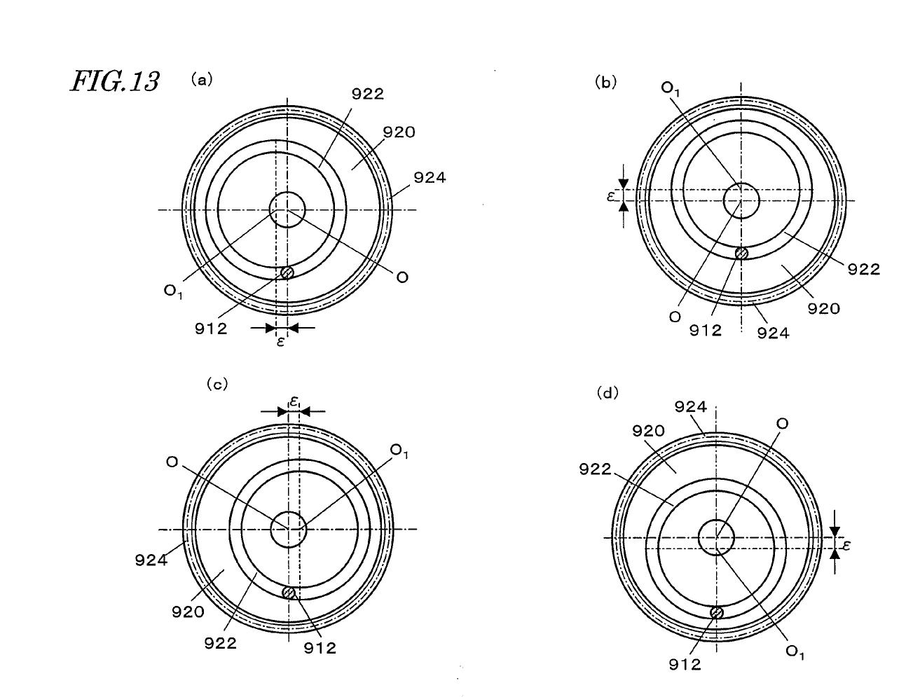

FIG. 10 is a partially cutaway cross-sectional view illustrating the internal configuration of the measuring section 510. FIG. 11 is a cross-sectional view thereof as viewed on the plane A-A shown in FIG. 10. FIG. 12 is a cross-sectional view thereof as viewed on the plane B-B shown in FIG. 10. And FIG. 13 shows plan views illustrating an example of a cam gear portion as viewed from a cam portion.

As shown in FIG. 10, inside the inserting portion 104 of the measuring section 510, arranged is an optical waveguide tube 710 including a rectangular cylindrical portion 712 with a rectangular profile and a circular cylindrical portion 714 with a circular profile. And inside this optical waveguide tube 710, an optical waveguide 716 runs through the rectangular cylindrical portion 712 and the circular cylindrical portion 714. Furthermore, inside the optical waveguide tube 710 and outside the optical waveguide 716, first and second acoustic waveguides 141 and 142 are arranged so as to define a tilt angle with respect to the center axis 718 of the optical waveguide 716.

One end of the optical waveguide tube 710 with the circular cylindrical portion 714 is extended to the vicinity of the end of the inserting portion 104 to be introduced into the acoustic foramen. On the other hand, the other end of the optical waveguide tube 710 with the rectangular cylindrical portion 712 is connected to the sensing block housing 720 to hold the sensing block 512.

Inside the sensing block housing 720, the sound source 143, the microphone 144, the chopper 118 and the infrared sensor 108 are fixed and the optical filter wheel 106 is supported rotatably.

Specifically, the chopper 118, the optical filter wheel 106 and the infrared sensor 108 are arranged inside the sensing block housing 720 such that when the inserting portion 104 is introduced into the acoustic foramen, the infrared radiation that has been incident through the end of the inserting portion 104 passes through the optical waveguide 716 inside the optical waveguide tube 710 and reaches the infrared sensor 108 by way of the chopper 118 and the optical filter wheel 106.

Also, those members are arranged such that the acoustic wave produced by the sound source 143 is transmitted through the first acoustic waveguide 141 into the acoustic foramen and that the wave is reflected back from inside the acoustic foramen toward the microphone 144 by way of the second acoustic waveguide 142.

As shown in FIGS. 10 and 11, the rectangular cylindrical portion 712 of the optical waveguide tube 710 is surrounded with a first supporting member body 742 with a cuboid hollow portion, which is further surrounded with a second supporting member body 752 that also has a cuboid hollow portion.

At the portion including the plane A-A shown in FIG. 10, a first pivot 812 that is secured to the first supporting member body 742 is fitted rotatably into a first pivot hole 810 that has been cut through the rectangular cylindrical portion 712 of the optical waveguide tube 710 as shown in FIG. 11. With this arrangement, the rectangular cylindrical portion 712 of the optical waveguide tube 710 can turn around the first pivot 812 inside the first supporting member body 742.

In addition, a second pivot 822 that is secured to the second supporting member body 752 is fitted rotatably into a second pivot hole 820 that has been cut through the first supporting member body portion 742. With this arrangement, the first supporting member body 742 can turn around the second pivot 822 inside the second supporting member body 752.

As shown in FIG. 10, the second supporting member body 752 is secured to a supporting member body 730, which is further secured to the inserting portion 104.

With this arrangement, the first supporting member body 742 connected to the optical waveguide tube 710 can change its tilt angles with respect to the inserting portion 104 by turning around the second pivot 822 and the optical waveguide tube 710 can change its tilt angles with respect to the inserting portion 104 by turning around the first pivot 812 that intersects with the second pivot 822 at right angles. The sensing block housing 720 is connected to the end of the optical waveguide tube 710 with the rectangular cylindrical portion 712, and therefore, moves along with the optical waveguide tube 710.

Next, the specific structures of the respective supporting members will be described. As shown in FIGS. 10 and 12, the second supporting member body 752 that is secured to the supporting member body 730 and a second supporting member's side plate portion 754 that is secured to the second supporting member body 752 together form a second supporting member 750. On the other hand, the first supporting member body 742 and a first supporting member's side plate portion 744 that is secured to the first supporting member body 742 together form a first supporting member 740.

Next, a structure for changing the tilt angles of the optical waveguide tube 710 will be described in detail with reference to FIGS. 10 through 13.

In the portion including the plane B-B shown in FIG. 10, the rectangular cylindrical portion 712 of the optical waveguide tube 710 includes a first cam follower 912 that passes the center 902 of the center axis of the optical waveguide 716. The first cam follower 912 is arranged such that the center axis of the first follower 912 is parallel to the first pivot 812.

A first cam gear shaft 910 is implanted into the first supporting member's side plate 744 and a first cam gear portion 920 is arranged so as to turn around the first cam gear shaft 910. Also, the first cam gear portion 920 has a first cam portion 922 in the shape of a groove. The first cam follower 912 is fitted into the first cam portion 922 so as to slide inside the first cam portion 922. Furthermore, a first worm wheel gear 924 is arranged beside the outer periphery of the first cam gear portion 920, and is engaged with a first worm gear 928 that is connected to a first drive motor 926. When the first drive motor 926 runs, its driving force is transmitted through the first worm gear 928 and the first worm wheel gear 924 to turn the first cam gear portion 920.

As the first cam gear portion 920 turns, the first cam follower 912 fitted into the first cam portion 922 moves along the groove of the first cam portion 922, thereby rotating the optical waveguide tube 710 around the first pivot 812.

In addition, in the portion including the plane B-B shown in FIG. 10, the first supporting member's side plate 744 also has a second cam follower 950, of which the center axis is parallel to the second pivot 822, as shown in FIG. 12.

A second cam gear shaft 960 is implanted into the second supporting member's side plate 754 and a second cam gear portion 970 is arranged so as to turn around the second cam gear shaft 960. Also, the second cam gear portion 970 has a second cam portion 972 in the shape of a groove. The second cam follower 950 is fitted into the second cam portion 972 so as to slide inside the second cam portion 972. Furthermore, a second worm wheel gear 974 is arranged beside the outer periphery of the second cam gear portion 970, and is engaged with a second worm gear 978 that is connected to a second drive motor 976. When the second drive motor 976 runs, its driving force is transmitted through the second worm gear 978 and the second worm wheel gear 974 to turn the second cam gear portion 970.

As the second cam gear portion 970 turns, the second cam follower 950 fitted into the second cam portion 972 moves along the groove of the second cam portion 972, thereby rotating the first supporting member 740 around the second pivot 822.

FIG. 13 shows plan views of the first cam gear portion 920 as viewed from over the first cam portion 922. FIGS. 13(a) through 13(d) show the respective positions of the first cam follower 912 that change every time the first cam gear portion 920 turns 45 degrees.

As shown in FIG. 13, the first cam portion 922 has a groove in the shape of a circle, of which the center is located at a point o₁ that has a degree of eccentricity E with respect to the center of rotation o of the first cam gear portion 920. That is why as the first cam gear portion 920 makes one turn, the first cam follower 912 fitted slidably into the first cam portion 922 moves vertically by 2ε.

Thus, in FIG. 10, as the first drive motor 926 runs, the first cam follower 912 arranged on the rectangular cylindrical portion 712 of the optical waveguide portion 710 can be moved vertically within a range of 2ε with respect to the first pivot 812 as a fulcrum. As a result, the other end 760 of the optical waveguide tube 710, opposite to the end connected to the sensing block housing 720, can be moved around the first pivot 812 as a fulcrum. By optimizing the distance from the first pivot 812 to the first cam follower 912 and the length of the optical waveguide tube 710 as measured from the first pivot 812 through the end 760, the range of movement of the end 760 of the optical waveguide tube 710 can be adjusted.

By performing a similar operation on the second cam gear portion 970, the end 760 of the optical waveguide tube 710 can be moved perpendicularly to the direction of movement of the end 760 of the optical waveguide tube 710 caused by the operation of the first cam gear portion 920.

Consequently, by controlling the operations of the first and second drive motors 926 and 976, the acoustic foramen can be scanned two-dimensionally to determine the direction that the end 760 of the optical waveguide tube 710 now faces.

Also, in this preferred embodiment of the present invention, drive motors are used to change the directions that the end 760 of the optical waveguide tube 710 faces and a worm gear system, which should be affected little by disturbance, is adopted. As a result, the direction that the end 760 of the optical waveguide tube 710 now faces can be adjusted highly precisely.

Hereinafter, it will be described how the biological information collecting system 500 of this preferred embodiment operates.

First, when the user presses the power switch 101 of the biological information collecting system 500, the power is turned ON inside the body 520 to get the biological information collecting system 500 ready to make measurements.

Next, the user holds the measuring section 510 with one of his or her hands to introduce the inserting portion 104 into his or her ear canal.

Subsequently, when the user presses the measuring start switch 103 of the biological information collecting system 500, the microcomputer 110 activates the frequency modulator 145, thereby producing first and second acoustic waves from the sound source 143. Then, based on the electrical signals representing the respective intensities of the first and second reflected waves that have been detected by the microphone 144, the microcomputer 110 determines whether or not the optical waveguide tube 710 in the inserting portion 104 faces the eardrum direction inside the acoustic foramen. This decision is made following the same procedure as in the third preferred embodiment, and the description thereof will be omitted herein.

If the buzzer 158 beeps to notify the user that the optical waveguide tube 710 in the inserting portion 104 has been introduced into the acoustic foramen improperly, or in a wrong direction, then the user turns the direction adjusting lever switch 522 to change the directions of the optical waveguide tube 710 inside the acoustic foramen such that the end face of the optical waveguide tube 710 inside the inserting portion 104 is opposed to the eardrum. In this case, the user may turn the direction adjusting lever switch 522 with one hand and hold the body section 520 with the other. Specifically, in the example illustrated in FIG. 8, settings may done such that the first drive motor 926 starts if the direction adjusting lever switch 522 is turned upward, the second drive motor 976 starts if the direction adjusting lever switch 522 is turned downward, and both of these two drive motors stop when the direction adjusting lever switch 522 is in the neutral position.

If the intensity of the first reflected wave detected by the microphone 144 is found greater than the first threshold value and if the intensity of the second reflected wave detected by the microphone 144 is found smaller than the second threshold value as a result of comparison made by the microcomputer 110, then it can be determined that the end face of the inserting portion 104 is opposed to the eardrum 202. Thus, it can be seen that the optical waveguide tube 710 inside the inserting portion 104 has been introduced properly into the acoustic foramen 200.

In that case, the microcomputer 110 controls the buzzer 158 to make the buzzer 158 emit a different sound from the alarm. On determining that the end face of the inserting portion 104 is opposed to the eardrum 202, the microcomputer 110 activates the chopper 118, thereby automatically starting to measure the infrared radiation emitted from the eardrum 202. By sounding the buzzer 158, the user can be notified that the inserting portion 104 has been introduced into the acoustic foramen 200 properly and that the measuring process has started.

The remaining infrared radiation measuring process step is quite the same as that of the third preferred embodiment and the description thereof will be omitted herein.

The biological information collecting device of this preferred embodiment includes an adjustment section for changing the directions of the optical waveguide inside the inserting portion. That is why even if it has been determined that the optical waveguide inside the inserting portion that has been introduced into the acoustic foramen does not face the eardrum direction, there is no need to move the measuring section itself but the direction of the optical waveguide can be adjusted just by turning the direction adjustment lever switch provided for the body portion.

In the preferred embodiment described above, the intensity of the infrared radiation is supposed to be measured with the measuring section 510 held in one hand. However, the present invention is in no way limited to that specific preferred embodiment. Alternatively, measuring section holding means for holding the measuring section onto the ear or the head may be provided for the measuring section and the intensity of the infrared radiation may be measured with the measuring section held onto the ear or the head with the measuring section holding means. Examples of the measuring section holding means include a clip for holding the measuring section onto the ear and a headband for holding the measuring section onto the head.

Also, in the second through sixth preferred embodiments of the present invention described above, electrical signals representing the intensities of acoustic waves that have been detected by the microphone 144 are supposed to be classified according to the frequency by the frequency analyzer 140 and then output to the microcomputer 110. However, the present invention is in no way limited to those specific preferred embodiments. For example, if a microcomputer with a fast Fourier transform function is used as the microcomputer 110, the electrical signals representing the respective intensities of the acoustic waves that have been detected by the microphone 144 can be classified according to the frequency by the microcomputer itself without using the frequency analyzer 140.

Furthermore, in the third through sixth preferred embodiments described above, the intensities of the first and second acoustic waves are defined to be equal to each other. However, the present invention is in no way limited to those specific preferred embodiments. Alternatively, the intensity of the first acoustic wave may be greater than that of the second acoustic wave. Conversely, the intensity of the second acoustic wave may be greater than that of the first acoustic wave.

### Embodiment 7

Hereinafter, a biological information collecting device as a seventh preferred embodiment of the present invention will be described.

The appearance of the biological information collecting device of this preferred embodiment is the same as that of the biological information collecting device 210 of the second preferred embodiment described above, and the description thereof will be omitted herein.

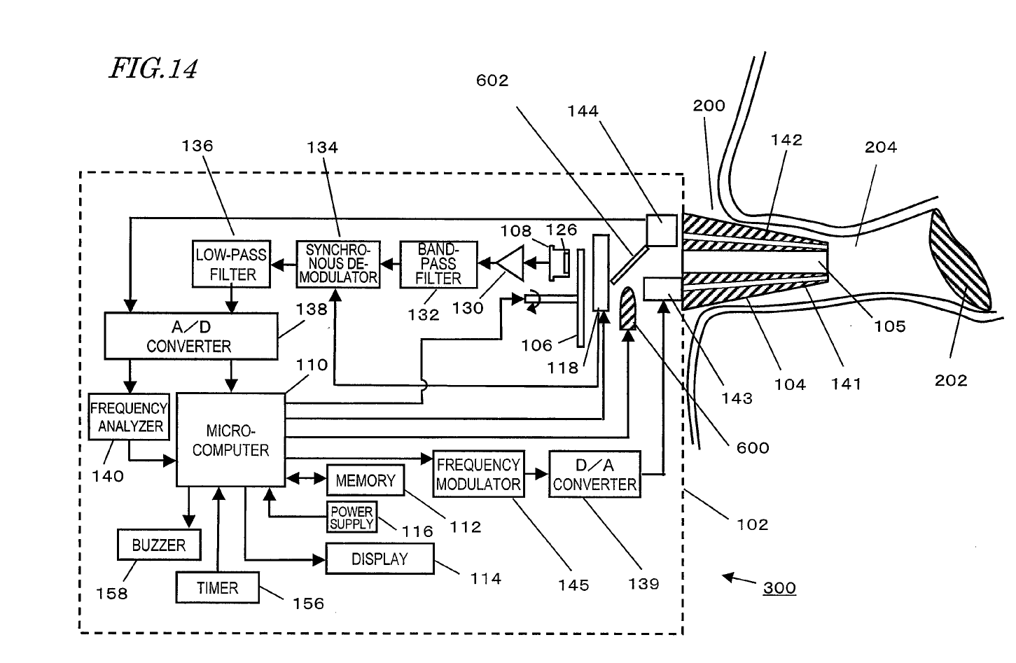

Next, the internal body configuration of the biological information collecting device as the seventh preferred embodiment of the present invention will be described with reference to FIG. 14, which illustrates a configuration for the biological information collecting device 300 of the seventh preferred embodiment.

Unlike the biological information collecting device 100 of the third preferred embodiment described above, the device 300 of this preferred embodiment further includes an infrared radiation source 600 for emitting an infrared radiation and a half mirror 602 inside the body of the biological information collecting device 100. Other than that, the configuration of this preferred embodiment is quite the same as that of the biological information collecting device 210 of the second preferred embodiment and the description thereof will be omitted herein.

The infrared radiation source 600 emits infrared radiation to irradiate the eardrum 202. The infrared radiation that has been emitted from the infrared radiation source 600 and then reflected from the half mirror 602 is guided through the optical waveguide 105 to enter the ear canal 204 and irradiate the eardrum 202. The infrared radiation that has reached the eardrum 202 is reflected from the eardrum 202 back toward the biological information collecting device 100. This infrared radiation is guided through the optical waveguide 105 again, transmitted through the half mirror 602 and the optical filter wheel 106, and then detected at the infrared sensor 108.

The intensity of the light reflected from the eardrum 202 to be detected in this preferred embodiment is calculated as the product of the reflectance given by Equation (8) and the intensity of the infrared radiation impinging on the eardrum 202. As the biological constituent changes its concentrations, the refractive index and the extinction coefficient of the organism change. That is why the biological constituent concentration can be obtained by measuring the intensity of the light reflected from the eardrum 202. The reflectance is normally as small as about 0.03 in the infrared range of the spectrum and depends very little on the refractive index and the extinction coefficient of the organism as can be seen from Equation (8). Thus, the reflectance hardly changes even when the biological constituent concentration varies. For that reason, the intensity of the infrared radiation emitted from the infrared radiation source 600 is preferably increased such that the intensity of the light reflected from the eardrum 202 varies more significantly with the biological constituent concentration.

As the infrared radiation source 600, any known light source may be used without restriction. For example, a silicon carbide light source, a ceramic light source, an infrared LED, or a quantum cascade laser may be used.

The half mirror 602 has the function of splitting an infrared radiation into two bundles of rays. The half mirror 602 may be made of ZnSe, CaF₂, Si, Ge or any other suitable material. Furthermore, to control the transmittance and reflectance of the infrared radiation, the half mirror 602 is preferably coated with an antireflection film.

In the memory 112, stored is correlation data showing the correlation between the respective signal values of the electrical signals representing the intensities of the infrared radiations that have been transmitted through the first and second optical filters 122 and 124 and the biological constituent concentration. The correlation data may be obtained in the following manner, for example.

First, as for a patient with a known biological constituent concentration such as a blood glucose level, the infrared radiation that has been emitted from the infrared radiation source 600 toward his or her eardrum and then reflected from the eardrum has its intensity measured. In this case, electrical signals representing the intensities of infrared radiations falling within the wavelength ranges to be transmitted by the first and second optical filters 122 and 124 are obtained. By making such measurement on a number of patients with mutually different biological constituent concentrations, multiple sets of data, each including the electrical signals representing the intensities of infrared radiations falling within the wavelength ranges to be transmitted by the first and second optical filters 122 and 124 and their associated biological constituent concentrations, can be collected.

Next, by analyzing these data sets that have been collected in this manner, correlation data is obtained. For example, a multivariate analysis is carried out by either a multiple regression analysis such as partial least squares regression (PLS) method or a neural network method on the electrical signals representing the intensities of infrared radiations falling within the wavelength ranges to be transmitted by the first and second optical filters 122 and 124 and their associated biological constituent concentrations. As a result, a function showing a correlation between the electrical signals representing the intensities of infrared radiations falling within the wavelength ranges to be transmitted by the first and second optical filters 122 and 124 and their associated biological constituent concentrations can be obtained.

Also, the first optical filter 122 may have such a spectral characteristic as to transmit infrared radiation falling within a measuring wavelength range and the second optical filter 124 may have such a spectral characteristic as to transmit infrared radiation falling within a reference wavelength range. In that case, the difference between the signal values of the electrical signals representing the intensities of infrared radiations falling within the wavelength ranges to be transmitted by the first and second optical filters 122 and 324 may be calculated, and the correlation between that difference and its associated biological constituent concentration may be obtained as the correlation data by performing a linear regression analysis such as a minimum square method, for example.

Hereinafter, it will be described with reference to FIGS. 3, 5 and 14 how the biological information collecting device of this preferred embodiment operates.

First, when the user presses the power switch 101 of the biological information collecting device 100, the power is turned ON inside the body 102 to get the biological information collecting device 100 ready to make measurements.

Next, the user holds the body 102 with his or her hand to introduce the inserting portion 104 into his or her ear canal 204 as shown in FIG. 14.

Subsequently, when the user presses the measuring start switch 103 of the biological information collecting device 100 with the biological information collecting device 100 held at the position where the outside diameter of the inserting portion 104 gets equal to the inside diameter of the acoustic foramen 200, the microcomputer 110 activates the frequency modulator 145 as in the third preferred embodiment described above, thereby producing first and second acoustic waves from the sound source 143. The frequencies, intervals, intensities and so on of the first and second acoustic waves are the same as the ones already described for the third preferred embodiment. The procedure to make the decision is also the same as that of the third preferred embodiment, and the description thereof will be omitted herein.

On finding the inserting portion 104 facing the eardrum 202, the microcomputer 110 turns ON the power of the infrared radiation source 600. As a result, the infrared radiation that has been emitted from the infrared radiation source 600 toward the eardrum 202 is reflected from the eardrum 202. Consequently, the infrared radiation radiated from the eardrum 202 starts to be measured.

On sensing, by reference to the clock signal supplied from the timer 156, that a predetermined amount of time has passed since the measuring process was started, the microcomputer 110 controls the infrared radiation source 600 to cut off the infrared radiation. As a result, the measuring process ends automatically. At this point in time, by controlling the display 114 or the buzzer 158, the microcomputer 110 shows a message telling that the measuring process has ended on the display 114, makes the buzzer 158 beep or outputs a voice message or an alarm through a loudspeaker (not shown), thereby notifying the user of the end of the measuring process. On confirming that the measuring process has ended, the user removes the inserting portion 104 from his or her acoustic foramen 200.

Next, the microcomputer 110 reads correlation data, showing the correlation between the respective signal values of the electrical signals representing the intensities of the first and second infrared radiations that have been transmitted through the first and second optical filters 122 and 124 and the biological constituent concentration, from the memory 112, and converts the electrical signal supplied from the A/D converter 138 into a biological constituent concentration with reference to this correlation data. The biological constituent concentration thus calculated is presented on the display 114 eventually.

According to this preferred embodiment, by comparing the intensities of the first and second reflected waves to the first and second threshold values, respectively, the user can see more exactly what the inserting portion 104 introduced into the acoustic foramen 200 now faces. In addition, since the measurements can be made with the end face of the inserting portion 104, introduced into the acoustic foramen 200, opposed right to the eardrum 202, the biological information can be collected even more accurately.

The present invention can be used effectively to collect biological information non-invasively, e.g., measure a glucose concentration (i.e., blood glucose level), a hemoglobin concentration, a cholesterol concentration, a neutral fat concentration, a protein concentration or the concentration of any other chemical constituent included in an organism without collecting blood or to measure the body temperature as well.

While the present invention has been described with respect to preferred embodiments thereof, it will be apparent to those skilled in the art that the disclosed invention may be modified in numerous ways and may assume many embodiments other than those specifically described above. Accordingly, it is intended by the appended claims to cover all modifications of the invention that fall within the true spirit and scope of the invention.

What is claimed is:

1. A biological information collecting device comprising:
an infrared sensor for sensing an infrared radiation that has been emitted from inside an acoustic foramen;
an acoustic wave output section, which is arranged so as to emit an acoustic wave toward the field of view of the infrared sensor;
a computing section for deriving biological information based on the output of the infrared sensor;
an acoustic wave detector for detecting a reflected wave that has been produced by the reflection of the acoustic wave from inside the acoustic foramen;
a decision section for determining, based on a result of detection made by the acoustic wave detector, or not an eardrum falls into the field of view of the infrared sensor; and
a comparison section for comparing an intensity of the reflected wave, which has been detected by the acoustic wave detector, to a predetermined threshold value,
wherein with a result of comparison made by the comparison section also taken into consideration, the decision section determines whether or not the eardrum falls into the field of view of the infrared sensor.

2. The biological information collecting device of claim 1, wherein the acoustic wave output section includes
a sound source that emits the acoustic wave and
an acoustic waveguide for guiding the emitted acoustic wave through the acoustic foramen and toward the field of view of the infrared sensor.

3. The biological information collecting device of claim 1, further comprising an acoustic waveguide for guiding the reflected wave from inside the acoustic foramen toward the acoustic wave detector.

4. The biological information collecting device of claim 1, further comprising a threshold value storage section for storing the predetermined threshold value,
wherein the predetermined threshold value has been determined in advance based on the intensity of the reflected wave, and
wherein the comparison section compares the intensity of the reflected wave that has been detected by the acoustic wave detector to the predetermined threshold value.

5. The biological information collecting device of claim 1, further comprising an alarm output section for outputting an alarm based on a result of comparison made by the comparison section.

6. The biological information collecting device of claim 1, wherein the acoustic wave output section emits the acoustic wave with at least one frequency selected from the frequency range of 1,000 Hz through 6,000 Hz.

7. The biological information collecting device of claim 1, wherein the acoustic wave output section emits the acoustic wave as a simple tone.

8. The biological information collecting device of claim 1, wherein the acoustic wave output section emits the acoustic wave with a constant intensity.

9. The biological information collecting device of claim 1, wherein the acoustic wave output section emits the acoustic wave with a constant frequency.

10. The biological information collecting device of claim 1, wherein the acoustic wave output section emits first and second acoustic waves to be reflected from an eardrum with mutually different reflectances, and
wherein the acoustic wave detector detects at least one of the reflected waves of the first and second acoustic waves.

11. The biological information collecting device of claim 10, wherein the decision section determines, based on the intensities of the reflected waves of the first and second acoustic waves, whether or not the eardrum falls into the field of view of the infrared sensor.

12. The biological information collecting device of claim 11, wherein the acoustic wave output section includes:
a sound source having the ability to emit one of the first and second acoustic waves selectively;
a first acoustic waveguide for guiding the first and second acoustic waves, emitted from the sound source, through the acoustic foramen and toward the field of view of the infrared sensor; and
a second acoustic waveguide for guiding the reflected waves of the first and second acoustic waves from inside the acoustic foramen toward the acoustic wave detector.

13. The biological information collecting device of claim 11, wherein the comparison section compares the respective intensities of the reflected waves of the first and second acoustic waves, which have been detected by the acoustic wave detector, to at least one threshold value, and
wherein with a result of comparison made by the comparison section also taken into consideration, the decision section determines whether or not the eardrum falls into the field of view of the infrared sensor.

14. The biological information collecting device of claim 13, further comprising a threshold value storage section for storing the at least one threshold value,
wherein the at least one threshold value includes first and second threshold values, and
wherein the comparison section compares the respective intensities of the reflected waves of the first and second acoustic waves that have been detected by the acoustic wave detector to the first and second threshold values, respectively.

15. The biological information collecting device of claim 13, further comprising a threshold value storage section for storing the at least one threshold value,
wherein the comparison section compares a differential value, showing a difference between the respective intensities of the reflected waves of the first and second acoustic waves, which have been detected by the acoustic wave detector, to the at least one threshold value.

16. The biological information collecting device of claim 13, further comprising an alarm output section for outputting an alarm based on a result of comparison made by the comparison section.

17. The biological information collecting device of claim 10, wherein the acoustic wave output section emits the first acoustic wave with at least one frequency selected from the frequency range of 20 Hz through 800 Hz and also emits the second acoustic wave with at least one frequency selected from the frequency range of 1,000 Hz through 6,000 Hz.

18. The biological information collecting device of claim 10, wherein the acoustic wave output section emits the first and second acoustic waves as simple tones.

19. The biological information collecting device of claim 10, wherein the acoustic wave output section emits the first and second acoustic waves, each of which has a constant intensity.

20. The biological information collecting device of claim 10, wherein the acoustic wave output section emits the first and second acoustic waves, each of which has a constant frequency.

21. The biological information collecting device of claim 1, further comprising a spectral element for subjecting the infrared radiation, which has been emitted from the acoustic foramen, to some spectral process.

22. The biological information collecting device of claim 11, further comprising a storage section for storing an output signal value of the infrared sensor in association with a result of decision made by the decision section.

23. A method for controlling the biological information collecting device of claim 22, the device further comprising a control section for controlling the infrared sensor, the acoustic wave output section, the acoustic wave detector, the computing section, the decision section and the storage section, the method comprising the steps of:
(a) getting the infrared radiation, emitted from inside the acoustic foramen, sensed by the infrared sensor;
(b) getting the first and second acoustic waves sequentially emitted from the acoustic wave output section;
(c) getting the reflected waves of the first and second acoustic waves detected by the acoustic wave detector;
(d) making the decision section determine, based on the respective intensities of the reflected waves of the first and second acoustic waves that have been detected by the acoustic wave detector, whether or not the eardrum falls into the field of view of the infrared sensor;
(e) storing the output signal values of the infrared sensor in the storage section in association with a result of decision made by the decision section; and
(f) getting an output signal value when it is determined by the decision section that the eardrum falls into the field of view of the infrared sensor read by the computing section from the output signal values stored in the output signal storage section and getting the biological information derived by the computing section based on the output signal value read.

24. A method for controlling the biological information collecting device of claim 22, the device further comprising a control section for controlling the infrared sensor, the acoustic wave output section, the acoustic wave detector, and the decision section, the method comprising the steps of:
(a) getting the first and second acoustic waves sequentially emitted from the acoustic wave output section;
(b) getting the reflected waves of the first and second acoustic waves detected by the acoustic wave detector;
(c) making the decision section determine, based on the respective intensities of the reflected waves of the first and second acoustic waves that have been detected by the acoustic wave detector, whether or not the eardrum falls into the field of view of the infrared sensor; and
if it has been determined in the step (c) that the eardrum falls into the field of view of the infrared sensor,
(d) getting sensing of the infrared radiation, emitted from inside the acoustic foramen, started by the infrared sensor.
